(19) United States
(12) Patent Application Publication
(10) Pub. No.: US 2008/0108138 A1
(43) Pub. Date: May 8, 2008

(54) Bioactive compositions and their use in cell patterning

(76) Inventors: Patrick Vermette, Sherbrooke (CA); Afra Hadjizadeh, Sherbrooke (CA)

Correspondence Address: OGILVY RENAULT LLP, 1981 MCGILL COLLEGE AVENUE, SUITE 1600, MONTREAL, QC, H3A2Y3, CA

(21) Appl. No.: 11/808,819

(22) Filed: Jun. 13, 2007

Related U.S. Application Data
(60) Provisional application No. 60/804,598, filed on Jun. 13, 2006.

(51) Int. Cl. C07C 53/00; C07K 16/00; C12N 5/00

(52) U.S. Cl. 435/404; 530/300; 562/512

(57) ABSTRACT

The present application relates to a bioactive composition comprising a arcuoid substrate and a polymeric film deposited on the surface of the substrate. The bioactive composition can control the patterning of a cell. The present application also relates to a method of modulating the patterning of a cell. This particular method comprises contacting the cell with the bioactive composition. The present application also relates to a method of producing the bioactive composition as well as a bioactive substrates produced by this particular method.

### FIELD OF THE INVENTION

The present invention relates to compositions and methods for controlling the patterning of cells in a three-dimensional environment. More particularly, the present invention relates to bioactive substrates capable of supporting the adherence of cells, the growth of cells, the migration of cells and the organization of cells with respect to one another and with respect to the longitudinal axis of the bioactive compositions.

### BACKGROUND OF THE INVENTION

It is generally recognized that the behavior of different cell types on polymeric materials depend largely on chemistry, topography and mechanical properties at the cell-biomaterial interface¹⁻⁶. Most cell types are known to orient and often move rapidly along fibres in the 5-108 micrometer range, which has been referred as to “contact guidance”^{5,7}. Surface topography, as well as fibre curvature, is of particular interest to modulate the patterning of cells such as their proliferation and migration in 3-D environments^{5,7,8}. A typical example of the effect of fibre curvature on the cell behaviour is that of endothelial cell coverage observed on vascular grafts made of poly(ethylene terephthalate) (PET) woven fibres⁸.

However, on contact with complex multicomponent biological media, spontaneous protein adsorption processes occur on the surfaces of synthetic biomaterials with heterogeneous biofilms formation typically comprising of various proteins. Much recent biomaterials research aims to overcome the lack of selectivity in this initial adsorption of biological molecules onto synthetic surfaces. A promising route towards achieving controlled and predictable biomedical responses is to pre-coat a biomaterial surface with a layer of a desired protein, or another biologically active molecule, that can exert predictable biomedical responses⁹. To modify biomaterial surfaces, bioactive ligands such as cell adhesion peptides may be either adsorbed or covalently grafted onto the surfaces of the biomaterials or included in their bulk composition¹¹.

In the last decades, scientists attempted to functionalize polymers with biologically active molecules such as fibronectin to obtain specific cell surface interactions. Only a fraction of the surface-immobilized proteins have proper orientation for cell adhesion due to their stochastic orientation on the surface. In addition, the surface properties of the materials such as surface charge, wettability and topography may influence the conformation and/or the orientation of the proteins. On hydrophobic surfaces they tend to maximize interaction with hydrophobic amino acid side chains. This can cause denaturation of the proteins or at least a different presentation of cell binding motifs¹¹. Most of the problems can be overcome by using cell recognition motifs such as small peptidic sequences. These small peptides exhibit higher stability in changing environmental conditions (such as pH-variation (e.g., vs. most adhesion proteins)), conformational shifting, and during storage. These small peptides are also easier to characterize and less expensive than most larger proteins. Because these peptides occupy a smaller volume, they can be packed with higher density on surfaces. This provides a chance to compensate for possibly lower cell adhesion activity. In addition, extracellular matrix (ECM) proteins contain normally many different cell recognition motives, whereas small peptides represent only one single motif. Therefore, they can selectively address one particular type of cell adhesion receptors. They are known to be stable against enzymatic degradation and should therefore exhibit excellent long-term stability¹¹.

On one hand, stable linking of biologically active molecules (e.g., RGD peptides) to a surface is essential to promote strong cell adhesion because formation of focal adhesions only occurs if the ligands withstand cells contractile forces. These forces are able to redistribute weakly adsorbed ligands onto a surface, which then lead only to weak fibrillar adhesions later on¹¹. Furthermore, cells can remove mobile integrin ligands by internalization¹¹. On the other hand, covalent immobilization of biologically active molecules onto polymer surfaces can often lead to a significant reduction in the activity of the immobilized molecules. To avoid this problem, a spacer layer is often inserted between the substrate surface and the bioactive molecule¹¹, preferably one with low non-specific protein adsorption properties (e.g., poly(ethylene oxide) (PEG), carboxy-methylated-dextrans (CMD), and partially amino-functionalized dextrans)¹².

It would be highly desirable to be provided with bioactive compositions that promote and control directional biological responses within three-dimensional environments.

### SUMMARY OF THE INVENTION

There is provided compositions and method for modulating the patterning of cells.

In a first aspect, there is provided a bioactive composition comprising an arcuoid substrate and a polymeric film linked to the surface of the arcuoid substrate, wherein said bioactive composition controls the patterning of a cell. In an embodiment, the arcuoid substrate is a fibre. In another embodiment, the arcuoid susbtrate comprises at least one of a polymeric material and a metal. In yet another embodiment, the polymeric material is at least one of poly(ethylene terephtalate) (PET), poly(tetrafluoroethylene) (PTFE), poly(epsilon-caprolactone) (PLC), polyethylene, polypropylene, silicone, polyvinyl chloride, polyurethane, polymethyl(methacrylate), ultra-high molecular weight polyethylene, poly(glycolic acid), poly(lactic acid), polystyrene, nylon, polycarbonate and polysulfone. In an embodiment, the diameter of the fibre is between about 1 μm to about 1 mm, and in still another embodiment, between about 50 μm to about 250 μm. In yet a further embodiment, the polymeric film is a cross-linked polymeric film. In still another embodiment, the cross-linked polymeric film comprises at least one of a n-heptylamine monomer and an acetyldehyde monomer. In yet another embodiment, the polymeric film is a deposited polymeric film. In still another embodiment, the polymeric film is cross-linked to the surface of the arcuoid substrate. In still a further embodiment, the polymeric film comprises a reactive group. In an embodiment, the thickness of the polymeric film is between about 2 nm to about 50 nm. In still another embodiment, the bioactive composition further comprises a biologically active entity linked to the polymeric film. In yet another embodiment, the biologically active entity is coupled to the polymeric film. In an another embodiment, the biologically active entity is linked to the polymeric film by an amide bond. In yet another embodiment, the biologically active entity comprises a peptidic sequence and/or is at least one of a matrix protein, a portion of a matrix protein, a variant of a matrix protein (e.g., fibronectin, laminin, vitronectin, etc.), a denatured matrix protein, an enzyme and a growth factor. In yet another embodiment, the biologically active entity is at least one of a RGD peptide and gelatin. In still another embodiment, the bioactive composition further comprises a spacer between the polymeric film and the biologically active entity. In this embodiment, the spacer is linked to the polymeric film and to the biologically active entity. In still a further embodiment, the spacer is coupled to the polymeric film and the biologically active entity. In yet another embodiment, the spacer is linked to the polymeric film and to the biologically active entity by an amide bond. In still another embodiment, the spacer comprises at least one of a carboxymethyl dextran, a poly(ethylene oxide), a partially amino-functionalized dextran and heparin. In an embodiment where the spacer comprises a carboxymethyl dextran, the molecular weight of the carboxymethyl dextran can be between about 50 kDa to about 250 kDa or about 70 kDa and the ratio of carboxylation of the carboxymethyl dextran can be about 1:2.

In yet another aspect, there is provided a method of controlling the patterning of a cell. In an embodiment, the method comprises contacting the cell on the bioactive composition described herein, thereby modulating the patterning of the cell.

In a further aspect, there is provided a method of modulating cell patterning properties of an arcuoid substrate. In an embodiment, the method comprises linking a polymeric film to the arcuoid substrate, thereby modulating the cell patterning properties of the arcuoid substrate.

In yet another aspect, there is provided a method of producing a bioactive composition capable of controlling the patterning of a cell. In an embodiment, the method comprises linking a polymeric film on the surface of an arcuoid substrate, thereby obtaining the bioactive composition. Various embodiments of the substrate and the fibre are described herein. In an embodiment, the polymeric film is linked to the surface of the substrate by deposition. In another embodiment, the polymeric film is deposited with radiofrequency glow discharge. Various embodiments of the polymeric films are described herein. In another embodiment, the method further comprises coupling a biologically active entity to the polymeric film. Various embodiments of the biologically active entity are described herein. In another embodiment, the method further comprises linking a spacer between the polymeric film and the biologically active entity, the spacer being linked to the polymeric film and to the biologically active entity. Various embodiments of the spacer are described herein.

In yet another aspect, there is also provided a bioactive composition produced by the methods described herein.

### BRIEF DESCRIPTION OF THE DRAWINGS

FIG. 1A illustrates a picture of the square holder used to keep polymer fibres (single filaments) within the plasma zone to modify fibre surfaces and subsequently to carry out XPS analyses.

FIG. 1B illustrates a picture of the circular holder used to keep single filaments within the plasma zone to modify fibre surfaces to be subsequently used in cell culture testing.

FIG. 2 illustrates a proposed chemical reaction that occurs at the surface of the fibre during its preparation according to one embodiment of the present invention. RGD peptides react via the N-terminus with carboxyl groups pre-activated with EDC and NHS to generate an active ester.

FIG. 3A illustrates a schematic diagrams (not to scale) of the methods used for the covalent surface immobilization of GRGDS and GRGES peptides via an n-hepthylamine plasma polymer (HApp) and carboxy-methyl-dextrans (CMD) interlayers.

FIG. 3B illustrates the chemical sequence of the GRGDS and GRGES peptides.

FIG. 4 illustrates high-resolution XPS C 1s spectra of clean untreated PET fibres (FIG. 4A), PET fibres coated with HApp layer (FIG. 4B), HApp+CMD (70 kDa) with a ratio of 1 carboxyl group to 2 sugar units produced from a CMD solution of 2 mg/ml (FIG. 4C), and HApp+CMD+GRGDS (0.5 mg/ml) (FIG. 4D). The spectra have been displayed relative to each other in the Y-direction to allow comparison of peak positions.

FIG. 5 illustrates high-resolution XPS C 1s spectra of (O) PET+HApp+CMD (70 kDa, 1 mg/ml), (+) PET+HApp+CMD (70 kDa, 2 mg/ml), (x) PET+HApp+CMD (70 kDa, 2 mg/ml) following autoclaving, (−) PET+HApp+CMD (500 kDa, 1 mg/ml), and (*) PET+HApp+CMD (500 kDa, 2 mg/ml). CMD with a ratio of 1 carboxyl group to 2 sugar units was used. The different CMD coatings show similar C 1s spectra.

FIG. 6 illustrates endothelial cell adhesion (4 hours following cell seeding) on PET fibres coated by the different layers. Error bars represent one standard deviation. 70 kDa CMD, carboxylation degree of 1:2, and 2 mg/ml CMD solution concentration were used in these experiments. Note that, surface immobilization of CMD on HApp-coated PET monofilaments significantly reduced HUVEC adhesion (1.00±0.04 cells/mm²; p<0.05). HApp-coated fibres: 16±1 cells/mm² and GRGDS- or GRGES-bearing fibres (0.1 mg/ml GRGDS): 8.0±0.3 cells/mm², (0.5 mg/ml GRGDS): 21±4 cells/mm², (1 mg/ml GRGDS): 25±2 cells/mm²; (0.5 mg/ml GRGES): 1.0±0.2 cells/mm²-p<0.05).

FIG. 7 illustrates confocal microscopic observations of endothelial cells at day 4 on PET+HApp+CMD (70 kDa, carboxylation degree of 1:2, 2 mg/ml) (FIG. 7A), PET+HApp+CMD+GRGDS (0.5 mg/ml) (FIG. 7B), PET+HApp+CMD+GRGES (0.5 mg/ml) (FIG. 7C), and at day 8 on PET+HApp+CMD+GRGDS (0.5 mg/ml) (FIG. 7D). Endothelial cells were stained for nuclei with SYTOX Green Nucleic Acid Stain and for actin filaments with TRITC-phalloidin in FIGS. 7A, 7B, 7C, and with dil-acetylated LDL in FIG. 7D. Original magnification was 200×.

FIG. 8 illustrates images obtained by epifluorescence microscopy of the HUVECs stained for actin stress fibers (red) and vinculin (green). Original magnification was 600×.

FIG. 9 illustrates epifluorescence microscope images of HUVEC spreading and orientation along the PET fibre axis (A-D) compared to HUVEC spreading on the TCPS (F). Confocal microscope image of actin filament orientation along the fiber axis (F). Original magnification was 200×.

FIG. 10 illustrates high-resolution XPS C1s spectrum of PTFE fibre (FIG. 10A) and PTFE fibre coated with HApp (FIG. 10B).

FIG. 11 illustrates endothelial cell adhesion and confluency (4 days following cell seeding) on clean untreated PTFE fibres (monofilaments) (FIG. 11A), and the PTFE fibres bearing the different layers: +HApp (FIG. 11B), +HApp+CMD (FIG. 11C), +HApp+CMD+RGD (0.5 mg/ml) (FIG. 11D), and HApp+CMD+RGE (0.5 mg/ml) (FIG. 11E). Endothelial cells stained for nuclei with SYTOX™ Green Nucleic Acid Stain. Scale bar=200 μm.

FIG. 12 illustrates schematic diagrams of one embodied method used for the covalent surface immobilization of RGDS peptide via an acetaldehyde plasma polymer coating and carboxymethyl-substituted dextrans according to one embodiment of the invention.

FIG. 13 illustrates high-resolution XPS C1s spectrum of PET monofilament (FIG. 13A), PET+Aapp (FIG. 13B), PET+Aapp+PEI (FIG. 13C), PET+Aapp+PEI+CMD (FIG. 13D), and PET+Aapp+PEI+CMD+RGDS (FIG. 13E). The spectra have been displaced relative to each other in the Y-direction to allow comparison of peak position.

FIG. 14 illustrates high-resolution XPS C1s spectra of PCL membrane (FIG. 14A), PCL membrane coated with HApp (FIG. 14B).

FIG. 15 illustrates endothelial cell adhesion and spreading (4 days following cell seeding) on untreated PCL porous membrane and PCL porous membrane bearing HApp. PCL (FIG. 15A). +HApp (endothelial cells stained for nuclei with SYTOX™ Green Nucleic Acid) (FIG. 15B), +HApp (endothelial cells stained for actin filaments with NBD-phallacidin) (FIG. 15C). Fibroblast cells adhesion and spreading (2 days following cell seeding) on untreated PCL porous membrane and PCL porous membrane bearing the HApp. PCL (FIG. 15D), PCL+HApp (FIG. 15E). Original magnification was 4×.

FIG. 16 illustrates the morphology and orientation behaviours of endothelial cells on Polytetrafluoroethylene (PTFE, 200 μm in diameter) (FIG. 16A); endothelial cells stained for nuclei with SYTOX™ Green Nucleic Acid Stain and actin filaments with NBD-phallacidin at day 7. Scale bar=200 μm. Polyethylene terephthalate (PET, 100 μm in diameter) (FIG. 16B); endothelial cells stained for nuclei with SYTOX™ Green Nucleic Acid Stain and actin filaments with NBD-phallacidin at day 7. Scale bar=100 μm. Polyethylene terephthalate (PET, 70 μm in diameter) (FIG. 16C); endothelial cells stained for nuclei with hoechst and actin filaments with NBD-phallacidin at day 4. Scale bar=70 μm. Poly(epsilon-caprolactone) (PCL flat porous membrane) (FIG. 16D); endothelial cells stained for nuclei with SYTOX™ Green Nucleic Acid and actin filaments with NBD-phallacidin at day 4. Original magnification was 10×. Polyethylene terephthalate (PET, 70 μm in diameter) (FIG. 16E); endothelial cells stained for actin filaments with NBD-phallacidin at day 4. Original magnification was 20×.

FIG. 17 illustrates endothelial cell adhesion (FIG. 17A) and spreading (FIG. 17B) on gelatin-coated polyethylene terephthalate (PET, 100 μm) monofilaments. Endothelial cells stained for nuclei with Hoechst and actin filaments with NBD-phallacidin at day 7. Original magnifications were 10× and 20× respectively.

FIG. 18 illustrates procedures used in the two in vitro methods designed to guide micro-vessel network patterning. The two systems consisted to introduce endothelial cells (i.e., HUVECs) either present on the surface of fibres or by dispersing cells around cell-free fibres. Fibres were maintained parallel in a Teflon holder ring (FIG. 18A). In Method 1, HUVECs were seeded and grown on fibres until confluence. Cells attached to the fibres were stained for actin filaments, using TRITC-phalloidin, and counterstained with SytoxGreen to highlight the position of the nuclei (FIG. 18B). The holder was embedded in a fibrin gel on which a layer of fibroblasts was seeded and culture medium was finally added (FIG. 18C1). In Method 2, HUVECs and holders supporting cell-free fibres were sandwiched between two layers of fibrin gel (FIG. 18C2). In FIGS. 18D1 and 18D2, after 8 days of culture, micro-vessel-like structures were formed parallel to fibre axis. Sprouting from these structures was observed. Fiber diameter: 100 μm. The thick arrow shows fibres and narrow ones shows micro-vessels.

FIG. 19 illustrates in vitro directional micro-vessel formation along the cell-coated polymer fibres embedded in fibrin gel. By day 3-4, HUVECs formed cell strands as observed by phase contrast microscopy (left-hand column) and after dil-ac-LDL uptake (right-hand column) on bare PET fibres (FIG. 19A), PET+HApp (FIG. 19B), PET+HApp+CMD+gelatin (FIG. 19C), and PET+HApp+CMD+RGD peptide (FIG. 19D). Lumen can be seen between the two cell layers, more specifically when cells were separated from fibres as shown in FIG. 19D. Sprouting began to appear along the cell strands, particularly with the surface-modified fibres. Dil-Ac-LDL uptake shows sprouting of new micro-vessels into fibrin gel from the initially formed micro-vessel along fibre axis. Fiber diameter: 100 μm. The thick arrows show fibres and narrow ones show micro-vessels.

FIG. 20 illustrates progression of micro-vessel reorganization and network formation. By day 14, tube-like structures have extended in the fibrin and connections were established between the different micro-vessels. Phase-contrast images demonstrating the presence of a lumen within the micro-vessel along fibres axis (FIGS. 20A and 20F) and in the interconnecting network (FIG. 20B). Immunochemistry with Factor VII antibody on histological sections underlined the endothelial cells forming a lumen nearby fibres (asterisks) as observed in FIG. 20D (without primary antibody, only stained with counter-stained) and FIG. 20E (positive immuno-histochemistry). Fiber diameter: 100 μm. The thick arrows show fibres and narrow ones show micro-vessels.

FIG. 21 illustrates the effect of fibre-to-fibre distances over micro-vessel formation. Fibres were distanced by approximately 100 μm; two lumens were joined to each other (FIG. 21A). Fibres were distanced by less than 500 μm and show vascular connection (FIG. 21B). Fiber diameter: 100 μm. The thick arrows show fibres and narrow ones show micro-vessels.

FIG. 22 illustrates observation by epifluorescence of the culture system used in Method 2, in which HUVECs and cell-free fibres were sandwiched in fibrin gels. After 5 days of culture, cells stained with calcein AM did not show well formed micro-vessel-like structures in the absence of fibroblasts (FIG. 22A). Conversely, in the presence of fibroblasts, HUVECs stained with diL-Ac-LDL formed a micro-vessel network (FIGS. 22B-22D). Micro-vessels found around fibres were stained for actin filaments, using TRITC-phalloidin, and counterstained with SytoxGreen to highlight the position of the nuclei (FIG. 22E). The thick arrows show fibres.

FIG. 23 illustrates a schematic illustration of the micro-vessel network formed progressively in the fibrin constructs containing fibres. By 14-21 days, connections between micro-vessels were established. Adapted from http://classes.aces.uiuc.edu/AnSci312/Capilary/Angilect.htm.

### DETAILED DESCRIPTION OF THE PREFERRED EMBODIMENT

The present invention relates to compositions and methods for controlling cell patterning. The present invention also relates to methods of producing the compositions.

In a first aspect, the present invention provides a bioactive composition capable of controlling the patterning of a cell. As used herein, the term “patterning of a cell” relates to the ability of the composition to control the biological response of a cell adhered thereto and/or of a cell in the vicinity of cells adhered thereto. More particularly, the biological response of a cell (adhered to the bioactive composition or in the vicinity of the bioactive composition) relates to its ability to adhere to a specific substrate, to migrate on this specific substrate, to grow and divide, to grow into a differentiated cell, to express differentiation markers, to form differentiated structures, to respond to a biological stimulus, to communicate with neighboring cells, and/or to organize its cytoskeleton with respect to other cells or with respect to one of the axis of the bioactive composition, to express different sets of genes, to express different proteins, to bear different lipids or carbohydrate structure, to adopt different phenotypes, etc.

For example, the bioactive composition presented herein may be able to modulate how the cytoskeleton of a cell is organized with respect to the longitudinal axis of the fibre of the bioactive composition. As used herein, the term “cytoskeleton” is intended to refer to the internal structure responsible of the shape of the cell. When cells are detached from a solid substrate, they are usually assuming a spherical shape. Once cells adhere to a particular surface, the usually stretch to adopt an oval-like shape. The cytoskeleton of the cell is responsible for modifying the shape of the cell. When cells adopt an oval-like shape, the cytoskeleton stretches the cell along one preferred axis. Some bioactive composition allow the substantial alignment of the actin filaments of a cell parallel to the longitudinal axis of the bioactive composition and/or parallel to the cytoskeleton of neighboring cells. Other bioactive compositions allow the substantial alignment of the actin filaments of a cell perpendicular to the longitudinal axis of the bioactive composition and/or parallel to the cytoskeleton of neighboring cells. As used herein, the term “substantial alignment” is intended to characterize the fact that the majority of actin filaments of the cell are aligned with a component of the bioactive composition (e.g. the longitudinal axis) or with the cytoskeleton of neighboring cells.

According to another aspect, the present invention relates to bioactive compositions comprising an arcuoid substrate and a polymeric film. As used herein, the term “arcuoid substrate” is intended to mean a substrate having at least one arcuate face. In an embodiment, the susbtrate may be a sphere or a round cylinder, such as a fibre. The terms “fibre” and “fiber” are used herein interchangeably and relate to a three-dimensional round cylindrical substrate. In an embodiment, the term “arcuoid substrate” also excludes planar substrates having a regular (smooth) or irregular (rough) surfaces. Such planar substrates include, but are not limited to, membrane or the surface of conventional cell culture dishes used for growing adherent cells. In an embodiment, the substrate provides solid support for the cells. In another embodiment, when cells adhere to the substrate, the substrate should display low cytotoxicity towards the attached cells and/or low immunogenicity when implanted in vivo. In a further embodiment, the substrates should be compatible with conventional cell culture techniques. In an embodiment, the surface of the substrate may be regular (smooth) or irregular (rough). In yet another embodiment, the substrate itself does not necessarily substantially allow cellular adhesion, growth and/or differentiation.

In another embodiment, when the substrate is a fibre, the diameter of the fibre can be constant along its longitudinal axis. Fibres of different diameter can be used. In an embodiment, the diameter of the fibre is constant with respect to its longitudinal axis and is between 1 μm to 1 mm. In another embodiment, the diameter of the fibre may vary with respect to the longitudinal axis of the fibre. For example, the diameter at one end of the fibre may be different than the one at the other end of the fibre. As another example, the diameter of the fibre may also augment and diminish when moving along the longitudinal axis the fibre. In yet a further embodiment, the diameter of the fibre at one point along its longitudinal axis is between about 1 μm to about 1 mm. When the diameter of the fibre is modulated, it affects the alignment of the actin filaments of the adhered cell with respect to the longitudinal axis of the fibre. For example, when the diameter of the fibre is between about 150 μm and 250 μm, the alignment of the actin filaments of the adhered cells is heterogeneous (e.g. alignment is substantially parallel in some cells, substantially perpendicular in other cells or between parallel and perpendicular in other cells). In another example, when the diameter of the fibre is between about 100 μm and 150 μm, most of the cells are aligned parallel to the longitudinal axis of the fibre. In another example, when the diameter of the fibre is between about 50 μm and 100 μm, most of the cells are aligned perpendicular to the longitudinal axis of the fibre.

In an embodiment, the substrate is composed of polymeric material and/or a metal material. The polymeric material can be, but is not limited to poly(ethylene terephtalate) (PET), poly(tetrafluoroethylene) (PTFE), poly(epsilon-caprolactone) (PLC), polyethylene, polypropylene, silicone, polyvinyl chloride, polyurethane, polymethyl(methacrylate), ultra-high molecular weight polyethylene, poly(glycolic acid), poly(lactic acid), polystyrene, nylon, polycarbonate and polysulfone. In an embodiment, once linked to the surface of the substrate, the polymeric film does not substantially delaminates when it is placed in an aqueous environment.

In the bioactive composition described herein, a polymeric film is linked to the surface of the substrate. In an embodiment, the polymeric film is deposited on the surface of the substrate. In another embodiment, the polymeric film is cross-linked to the surface of the substrate. Without wishing to be bound to theory, the polymeric film is thought to allow the adherence of the cells and thereby allows the bioactive composition to control the patterning of the cell. In an embodiment, the polymeric film may be linked homogeneously on the surface of the substrate, thereby allowing cellular adherence and growth on the entire surface of the substrate. In another embodiment, the polymeric film may be linked on certain specific portions of the substrate, thereby allowing cellular adherence and growth only on these specific portions of the substrate.

Optionally, the polymeric film can be deposited and cross-linked concomitantly or independently.

In an embodiment, the polymeric film is composed of cross-linked monomeric subunits and therefore the polymeric film may be a cross-linked polymeric film. In an embodiment, the monomeric subunits can be cross-linked and/or deposited on the surface of the substrate using plasma polymerisation by a radiofrequency glow discharge. Applicant respectfully submits that extensive experimentations have been performed to deposit the polymeric film by plasma polymerisation. In an embodiment, once cross-linked and deposited on the surface of the substrate, the monomeric subunits should allow cellular attachment, growth, migration and/or differentiation. Therefore, the polymeric film composed of monomeric subunits should not be cytotoxic to cells adhered thereto, should promote cellular adhesion, migration, division, growth and differentiation. These monomeric subunits can be, for example, derived an ammoniacal monomer, a n-heptylamine monomer or an acetyldehyde monomer. In addition, depending on the monomer used, the polymeric film may comprise an ammonia polymer, a heptylamine polymer (HApp) or an acetyldehyde polymer (Aapp). In another embodiment, the polymeric film should contain a reactive group. Without wishing to be bound to theory, this reactive group can be used to link additional entities, such as a spacer or a biological active entity. In an embodiment, the polymeric film is a thin film and preserves substantially the diameter of the fibre and the topology of the fibre. In a further embodiment, the thickness of this polymeric film is between about 2 nm to about 50 nm. In yet another embodiment, the thickness of the polymeric film is superior to the “debye length” to avoid electrostatic interference with underlying layers.

In an embodiment, the bioactive composition may be produced and used in the shape of a single substrate (such as a single fibre) or may be connected to other substrates (such as other fibres) or other known biomaterial substrates. For example, the bioactive composition can be knit or woven with other substrates, parts of the bioactive composition may be glued, stapled or stitched to other biomaterial. When the bioactive composition is used in vivo, it may be glued, stapled or stitched to other biomaterial or directly on/in surrounding tissues or organs.

Optionally, the bioactive composition further comprises a biologically active entity. As used herein, the term “biological active moiety” or “biologically active moiety” relates to an entity capable of binding to the surface of the cell to promote its adherence to the bioactive composition. The biological active moiety may specifically bind specific structures on the surface of the cell (such as, for example integrins, glycoproteins, receptors, ion channels, etc.) or it may unspecifically bind to various structures on the surface of the cell. In an embodiment, the binding of the biologically active entity to the cell may provoke a cellular response (such as increasing of the cell attachment, the internalization of proteins, the reorganization of the cytoskeleton, differentiation of the adhered cells such as the formation of angiogenic sprouts, etc.). The biologically active entity may be linked or coupled directly to the polymeric film of the bioactive composition. When a biological active entity is linked or coupled directly to the polymeric film, precautions are taken not to alter the biological function of the entity. Alternatively, the biological active entity may be linked or coupled indirectly to the polymeric film of the bioactive composition. For example, the biologically active entity may be linked to a spacer molecule, such spacer molecule adapted to be linked or already linked to the polymeric film of the bioactive composition. In an embodiment, the link between the biological active moiety and the spacer or the polymeric film is a covalent link. In another embodiment, the link between the biologically active entity and the polymer film or the spacer may be a carboxyl group reacting with an amine group to form an amine bond, an ester bond, a thioester bond, an urea bond, a guanidine bond and/or a formamidine bond. In a further embodiment, the biological active moiety be grafted on the spacer or on the polymeric film.

Much like the deposited polymeric film, the biologically active entity may be grafted homogeneously on the surface of the polymeric film or it may be grafted heterogeneously. When the biologically active entity is grafted homogeneously, it may provide an homogeneous surface for cellular attachment. When the biologically active entity is grafted heterogeneously, some of the cells may adhere (or not) directly on the polymeric film and other cells may adhere (or not) to the linked biologically active entity.

The nature of the biological active moiety used in the bioactive composition will depend on the type of cells that need to be attached or grown on the substrate. The biologically active entity may be able to specifically bind to virtually all cell types and, alternatively, it may be able to bind to a specific cell type or only to a specific sub-population of a defined cell type. Therefore, by selecting a biologically active entity having defined properties with respect to cellular attachment and growth, one can select the kind of cells that will preferably bind, adhere, grow, migrate and/or differentiate on the bioactive composition.

In an embodiment, a single type of biologically active entity is present in the bioactive composition. In another embodiment, more than one type of biologically active entities are present in the bioactive composition. The latter can be useful in situations where two or more different types of cells specifically binding to distinct biologically active moieties need to be grown on the same bioactive composition. When two or more biologically active entities are used, they can be integrated homogeneously or heterogeneously in the bioactive composition, depending on the specific needs.

In an embodiment, the biologically active entity has a nucleotidic sequence, a polypeptidic sequence, a carbohydrate motif or a lipid moiety. For example, the biologically active entity may be a protein (such as a matrix protein (e.g., fibronectin, laminin, vitronectin, etc.), a portion thereof or a variant thereof; a glycoprotein, an enzyme and/or a growth factor such as VEGF and FGF), a peptide sequence, a lectin, an antibody or epitope recognizing fragment thereof, etc. In an embodiment, the biologically active entity may be derived from a microorganism (such as a virus, a bacteria, a fungus). Since some microorganisms can specifically bind to a very well defined structure on the surface of a cell, the microbial component responsible for the specific attachment to the cell may be used as a biologically active entity. In an embodiment, the biologically active entity may be derived from an animal or a human. In a further embodiment, the biologically active entity may contain a peptidic sequence (e.g. growth factor, enzyme, matrix protein, etc.). Since eukaryotic cells are usually embedded in a complex tridimensional matrix, the components of the tridimensional matrix may be used as a biologically active entity. In addition, since certain components of the matrix are present in virtually all matrices and other components are present in association with certain types of cells, matrix components may be selected for their binding specificity. Matrix components may be, for example, fibronectin, vitronectin, laminin, collagen, etc. Matrix components may be further altered to either increase or decrease their binding specificity (e.g. they may be shortened, lengthened, mutated, fused to another entity). For example, gelatin may be used as a biologically active entity. Since cells usually recognize small peptidic sequence in the matrix protein, peptide sequence may also be used as a biologically active entity. In an embodiment, the biologically active entity is the tripeptide RGD or a peptidic sequence containing this tripeptide.

When a biologically active entity is used in the composition, it may be necessary to insert a spacer between the polymeric film and the biologically active entity. Indeed, the immobilization of biologically active entity on a polymeric film can lead to a reduction in activity (e.g. binding capacity to the cells) of the immobilized biologically active entity. To circumvent this effect, a spacer molecule may be inserted between the polymeric film and the biologically active entity. In an embodiment, the spacer may have low non-specific protein adsorption properties. These spacers include, but are not limited to, carboxymethyl dextran, poly(ethylene oxide), partially amino-functionalized dextran, heparin, a polysaccharide and/or a phospholipid. In an embodiment, when carboxymethyl dextran is used as a spacer, its molecular weight is preferably between about 50 kDa to about 250 kDa and, in a further embodiment, about 70 kDa. In yet another embodiment, the ratio of carboxylation of the carboxymethyl dextran used in the bioactive composition is preferably about 1:2.

In an embodiment, the spacer used in the bioactive composition has low-fouling properties. This is of particular importance to reduce or eliminate the non-specific binding of proteins (such as serum proteins) to the bioactive composition. As such, a spacer with low-fouling properties will limit the attachment of non-specific binding proteins and/or facilitate the binding or coupling of biologically active entities.

In a further embodiment, for the bioactive compositions described herein, the cells should specifically bind to these compositions by a specific biosignal (usually present in the biologically active entity). To produce these bioactive compositions, a layer of a low-cell binding polymer (such as a low-fouling spacer) is first covalently linked to the substrate. In an embodiment, the low-cell binding layer limits non-specific cell attachment and cellular responses.

The bioactive compositions described herein can be applied to control the patterning of various cell types. In an embodiment, the cell is a prokaryotic cell, such as a bacterial cell or a fungal cell. In another embodiment, the cell is an eukaryotic cell, such as an animal cell, a human cell or a plant cell. For example, the eukaryotic cells may be derived from various organs and tissues. In yet another embodiment, the cell is an endothelial cell or a fibroblast. In an embodiment, the cells that can be used in conjunction with the bioactive composition is an adherent cell.

Since the bioactive compositions described herein modulate cellular adherence, growth, migration, cytoskeletal organization and/or positioning of cells with respect to one another or with respect to the longitudinal axis of the fibre, they are useful in controlling cell patterning. Because the pattern of cells is controlled by the nature of the bioactive composition, the bioactive compositions can be very useful. In an embodiment, the bioactive compositions can provide homogeneous cell cultures for in vitro screening procedures involving cell culture, such as highthrough-put screening procedures. In another embodiment, the bioactive compositions can be used to create in vitro organs or tissue in which cellular patterning is important for the functionality of these reconstructed organs of tissue. These in vitro organs or tissues can further be used for in vitro testing (e.g. response to a biological stimuli, screening of compound having a specific activity from a library of compounds), for the production of biologically active products or can also be used as replacement organ or tissue. In co-culture experiments, these bioactive compositions can be used to specifically modulate the responses of different cell types in different positions within a three-dimensional environment, thereby allowing the production of organized tissue substitutes consisting of more than one cell type.

According to a further aspect, there is provided a method of modulating the cell patterning properties of a substrate. This method comprises the linking of a polymeric film to the substrate, thereby modulating the cell patterning properties of the substrate.

In a further aspect, there is provided a method of controlling the patterning of a cell. The method comprises the step of contacting the bioactive composition described herein with the cell. Without wishing to be bound to theory, once in contact with the bioactive composition, the cell will adhere to the surface of the bioactive composition, and/or be attracted to the vicinity of the adhered cell. The cell may then migrate on the bioactive composition, grow, divide and/or differentiate. This method may be applied to any cell capable of adhering to the bioactive composition. In an embodiment, the polymeric film may be deposited by plasma polymerisation using a radiofrequency glow discharge. In yet a further embodiment, the plasma may be deposited with at least one of the following parameters: a frequency of about 50 kHz; a load power of about 80 W; an initial monomer pressure of between about 0.04 Torr to about 0.30 Torr; a deposition time of about 70 s; and a distance between the electrodes of about 10 cm. Various embodiments of the polymeric film that is being deposited on the substrate have been described above. Various embodiments of the substrate that can be used in this method have been described above. In an embodiment, a biologically active entity is linked to the polymeric film. Various embodiments of the biologically active entity have been described above. In an embodiment, the biologically active entity is linked or coupled directly to the polymeric film. In another embodiment, the biologically active entity is linked or coupled to a spacer which has been previously linked or coupled to the polymeric film. Various embodiments of the biologically active entity and of the spacer have been described above.

In another aspect, there is also provided a bioactive substrate produced by the method described above.

More specifically, in an embodiment a thin polymeric interfacial bonding layer bearing amine groups was coated onto 100-μm diameter PET fibres by radiofrequency glow discharge (RFGD) deposition. Plasma polymerization of n-heptylamine was carried out in a custom-built plasma reactor. This method is known to deposit a cross-linked organic thin film with functional groups. Afterwards, carboxy-methyl-dextran (CMD) was covalently grafted onto amine groups-coated fibres using water-soluble carbidiimide chemistry. RGDS peptides were covalently immobilized onto CMD-coated fibres by using also carbodiimide chemistry. CMD is a polysaccharide with carboxyl groups that can be used as a spacer to covalently link bioactive compounds and as a low-fouling layer to limit non-specific protein adsorption and cell attachment onto medical devices³.

In vivo adhesion events occur three-dimensionally, where cells attach to surrounding three-dimensional mesh-like fibres of the ECM, rather than onto two-dimensional surfaces. Artificial 2-D substrates made of plastic or glass that are often used in cell culture experiments misrepresent findings by forcing cells to adjust to artificially flat and rigid surfaces. Also, these are of little use in the culture of tissue mass. Cell-matrix interactions mediate many physiological responses including the regulation of cell growth, cell migration, cell differentiation, cell survival, tissue organization, and matrix remodeling, to name a few. The types of cell-matrix interactions organized by integrins in vitro and the signals they transduce are strongly affected by the flat rigid surfaces of tissue culture dishes; therefore, a closer approximation to in vivo environments should be attained by growing cells in three-dimensional environment.

Several techniques have been developed to generate microscopic patterns of biomolecules on different materials surfaces. Such chemical patterns have been successfully used as model surfaces for biorelated studies to investigate cell-surface interactions. Patterns of cell adhesive and non-adhesive molecules can constrain cell adhesion to specific areas to address a great number of cell biological questions such as cell adhesion and cell spreading, cell migration, cell mechanics and cell-cell communications. A second important area that has been extensively studied is the influence of different micro- and nano-topographies on cell behaviour and cell guidance. Many studies published in the last decades have aimed at understanding the effect of topographical and chemical effects on the cell interaction at the interface between biomaterials and biological media and the subsequent tissue formation. Few studies, however, have covered the biological performance of surfaces that exhibit geometrically well-defined, and simultaneously present biochemical cues in a controlled manner.

In the present application, bioactive polymer fibres have been produced. These fibres can control directional biological responses within 3-D environments by combining the topographical structure (defined at the nanoscopic scale) with modification to achieve spatial control of cell-adhesive peptides and for subsequent 3-D culture of cells in a biochemically defined environment.

The present invention will be more readily understood by referring to the following examples which are given to illustrate the invention rather than to limit its scope.

### Example I

### Production of Biologically-Active Pet Fibres

Materials used. Commercially available 100-μm diameter monofilaments made of poly(ethylene terephthalate) (PET, cat. #ES305910, Good fellow, Devon, USA) were used. PET fibres have been selected because of their commercial availability in monofilaments, their amenability to sustain the multi-step surface modification used in this study, their biocompatibility, and their resistance towards standard autoclaving sterilization methods.

Borosilicate glass substrates were purchased from Chemglass (Vineland N.J., USA). N-heptylamine (99% purity) used in plasma polymerisation was obtained from Sigma-Aldrich (cat. #51958, Saint-Louis, Mo., USA). Dextrans of 70 and 500 kDa molecular weights were purchased from Amersham Bioscience (cat. #US14495, Upsala, Sweden). Dextrans were carboxy-methylated to ca. 50% following a procedure similar to the work of Löfas and Johnsson. Briefly, CMD with ratios of carboxyl groups to anhydroglycopyranoside rings of 1:2 were prepared by dissolving 10 g of dextran in 50 ml of 2M NaOH containing 1M bromoacetic acid. The solution was stirred overnight, dialysed against water for 24 h, then against 0.1M HCl for 24 h, and finally for 24 h against water. The solution was then lyophilised and stored at 4° C. until used. The degree of carboxylation was assessed using NMR. The experimentally determined ratios were ca. 1 carboxyl groups per 2 sugar units. GRGDS (cat. #44-0-23) and GRGES (cat. #44-0-51) peptides were purchased from American Peptide Company (Sunnyvale, USA).

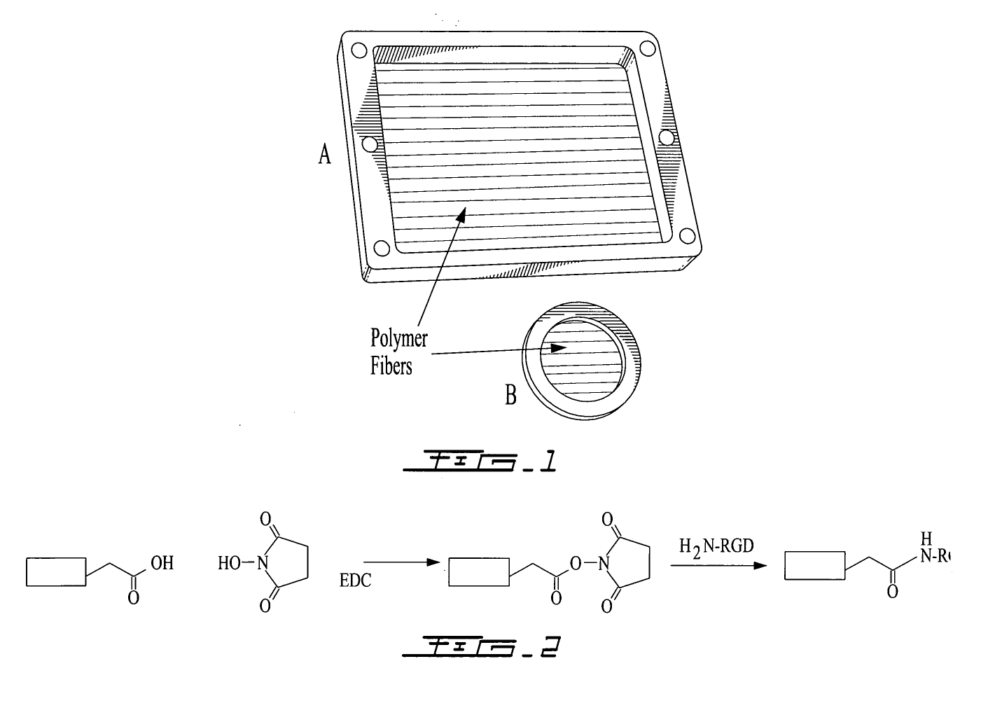

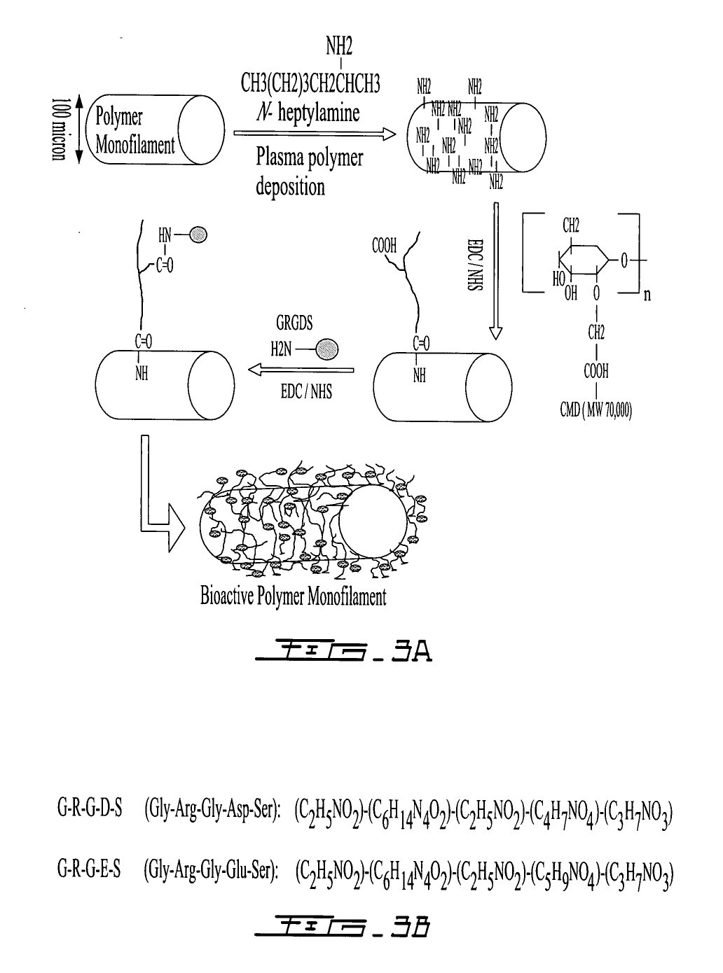

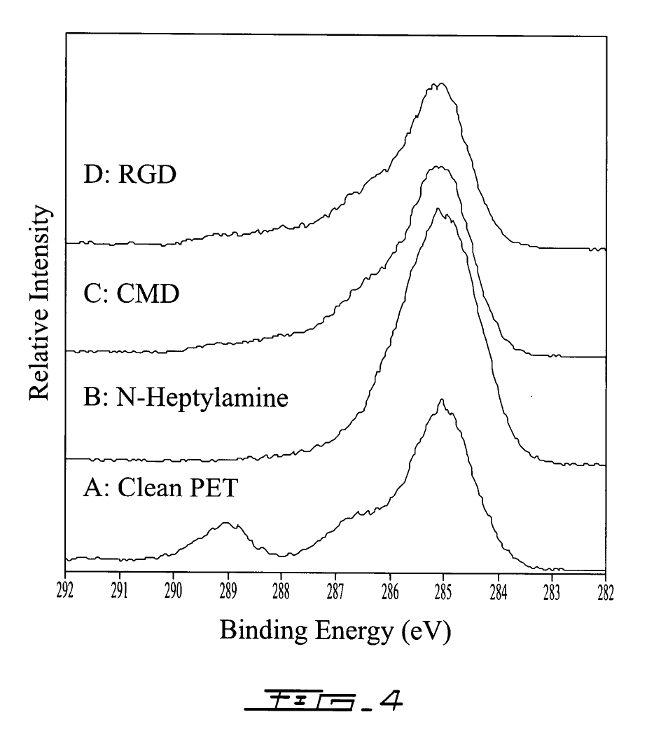

Preparation of PET fibres. PET fibres were fixed onto non-conductive 3-mm thick Teflon frames [5×7 cm square holder (FIG. 1A) and 2-cm diameter circular holder (FIG. 1B)] to hold fibres steady in the plasma zone. The square frame was used to facilitate sample preparation (5-cm length fibres) for XPS characterization. The circular frame was designed for cell cultures in 12-multiwell plates. Fibres secured onto the frames were cleaned by an overnight incubation into a surfactant solution (RBS™ Detergent 35, Pierce Biotechnology, Rockford, Ill., cat. #27952). Then, fibres were sonicated for 10 minutes in RBS solution followed by 10 minutes in ethanol (ACS grade). Scanning electron microscope (SEM) analyses of the PET fibres exposed to such a sonication procedure revealed no damage to the PET monofilaments. Fibres were thoroughly rinsed under a flow of Milli-Q water (with a resistivity of not less than 18.2 MΩ*cm) and blow-dried using 0.2-μm filter-sterilized compressed air.

Plasma polymerization. PET fibres firmly fixed to the holders were first activated using plasma Radio Frequency Glow Discharge (RFGD) deposition to obtain a cross-linked polymeric thin film with surface reactive groups (FIG. 2) onto which further layers could be immobilized by aqueous phase chemistry. The reactor employed for plasma polymerisation is a custom-built batch reactor in which plasma is produced in vacuum by a radio frequency supply. The holders bearing the fibres were placed on the lower circular electrode, which has a diameter of 9.5 cm. Deposition of thin plasma polymer films was carried out from vapour of n-heptylamine as described elsewhere.¹³ Parameters selected for the plasma deposition of n-heptylamine were a frequency of 50 kHz, a load power of 80 W, and an initial monomer pressure of 0.040 Torr. Deposition time was set to 70 s and the distance between the electrodes was set at 10 cm.

CMD immobilization onto plasma-modified PET fibres. CMD was attached directly to n-heptylamine plasma polymer (HApp)-coated fibres using water soluble carbodiimide chemistry. One (1) and 2 mg/ml CMD solutions were prepared in Milli-Q water. Once dissolved, 19.2 mg/ml of 1-ethyl-3-(3-dimethylaminopropyl) carbodiimide (EDC) and 11.5 mg/ml of N-hydroxysuccinimide (NHS) were added to the CMD solutions. Holders bearing HApp-coated monofilaments were then immersed in this solution and let to react overnight at room temperature under vigorous agitation. To remove any non-covalently attached CMD, the fibres were rinsed for 24 hours under vigorous agitation in a 1M NaCl solution, followed by an immersion in Milli-Q water for 24 hours under agitation at room temperature before being again rinsed in Milli-Q water and subsequently used.

RGD peptide grafting onto CMD-coated fibres. GRGDS and GRGES immobilization was carried out according to a sequential procedure described in the prior art. To avoid any potential damages of the GRGDS and GRGES peptides during the sterilization of the PET monofilaments prior to cell culture, peptide immobilization was performed under sterile conditions using pre-sterilized CMD-covered fibres. CMD-coated fibres were placed in Milli-Q water and autoclaved at 121° C. for 25 minutes. Then, sterile fibres were immersed into filter-sterilized (low-binding 0.22-μm syringe filters, Millipore corporation, Bedford, Mass., USA) solutions of EDC (19.2 mg/ml) and NHS (11.5 mg/ml) in PBS (1×, pH 7.4) at room temperature for 4 hours, followed by rinsing with PBS (pH 7.4; 2×15 minutes). The fibres were then incubated into a filter-sterilized (0.22-□m) peptide solution of 0.1, 0.5 or 1 mg/ml GRGDS or GRGES made in PBS (pH 8) for 2-3 h at room temperature. After being rinsed with PBS (pH 8, 3×15 minutes) to remove unbounded free peptides, fibres were left to dry under sterile hood. The advantage of this sequential procedure is that it limits intra- and/or intermolecular condensation of the RGD peptides. Using this two-step procedure, the carboxyl groups of the aspartate side chain is not activated and is therefore not available for coupling. Also, protonation of the arginine side chain in water is nearly abolished.

Surface characterization of polymer fibres by X-ray photoelectron spectroscopy (XPS). XPS analyses were performed using an AXIS HS spectrometer (Kratos Analytical) equipped with a monochromatic AI Kα source at a power of 120 W. The total pressure in the main vacuum chamber during analysis was typically 2×10⁻⁸ mbar. Elements present were identified from survey spectra. For further analysis, high-resolution spectra were recorded from individual peaks at 40-eV pass energy. Atomic concentrations of each element were calculated by determining the relevant integral peak area (using a Shirley-type background) and applying the sensitivity factors supplied by the instrument manufacturer. The random error associated with elemental quantification has been determined, for this instrument, to be 1-2% of the absolute values for atomic percentages>5%. A value of 285 eV for the binding energy of the main C 1s component (CHx) was used to correct for charging of specimens under irradiation.

Assuming a value of ≈3 nm for the electron attenuation length of a C 1s photoelectron in a polymeric matrix, this translates into an approximate value for the XPS analysis depth (from which 95% of the detected signal originates) of 10 nm when recording XPS data at an emission angle normal to the surface.

### Example II

### Cell Culture

Cell culture. Human umbilical vein endothelial cells (HUVECs) were isolated from human umbilical cord veins. HUVECs were cultured in M199 culture medium (cat. #M5017, Sigma-Aldrich, Saint-Louis, Mo., USA containing 2.2 mg/ml sodium bicarbonate (Fisher, Fair Lawn, USA), 90 μg/ml sodium heparin (cat. #H1027, Sigma-Aldrich, Saint-Louis, Mo., USA), 100 U/100 μg/ml penicillin/streptomycin (cat. #15140-122, Invitrogen Corporation, NY, Grand Island, USA), 10% fetal bovine serum (FBS, cat. #F1051, Sigma-Aldrich, Saint-Louis, Mo., USA), 2 mM L-Glutamine (cat. #25030149, Invitrogen Corporation, NY, Grand Island, USA), and 25 μg/ml endothelial growth factor supplement (ECGS, cat. #356006, BD Biosciences, San Jose, Calif., USA).

Cell seeding onto surface-modified fibres. To evaluate the effect of surface-modified polymer fibres on HUVEC responses and patterning, fibres were positioned in parallel direction onto a circular Teflon holder (2 cm in diameter, FIG. 1B). On this holder, fibres were distanced of approximately 0.5 to 2 mm from each other. Fibres were then surface-modified and subsequently sterilized directly onto the holders; non-coated PET fibres, HApp- and CMD-coated fibres were placed in Milli-Q water and autoclaved at 121° C. for 25 minutes. Three specimens of uncoated cleaned PET monofilaments and PET monofilaments coated with 1) HApp, 2) HApp+CMD, 3) HApp+CMD+GRGDS, 4) HApp+CMD+GRGES layers were investigated. Holders bearing the fibres were placed into wells of 12-well tissue culture polystyrene (TCPS) plates, then rinsed in sterile PBS (pH 7.4), and incubated in M199 culture medium for 1-2 hours to keep fibres hydrated until cells were seeded. Then, 1 ml of M199 culture medium containing 1×10⁶ cells were directly poured into the well containing the fibre holder. Cells of passages 3 to 5 were used in all experiments. Fibres were incubated for 1 hour and agitated. Afterward, the holders bearing the fibres were transferred into new 12-well TCPS plates containing fresh M199 culture media and incubated in a CO₂ incubator at 37° C. and 5% CO₂. Controls were carried out with the same cells grown directly on the plastic surface of the 12-multiwell TCPS plates.

### Example III

### Assessment of Biological Activity of the Fibres

Cell attachment assessment. Cell attachment assessment was performed following a 4-hour incubation period. The culture media were removed from the wells and cells were incubated with Hoechst 33258 (cat. #H1398, Molecular Probes Inc., Burlington, Canada) at a concentration of 10 μg/ml in a fresh M199 culture medium for 30 minutes. Samples were then observed under an inverted microscope (Nikon) using fluorescence and phase contrast imaging. Images were taken with a 10× lens in conjunction with a digital CCD camera (Regita 1300R, QIMAGING, Burnaby, Canada) and an imaging software (SimplePCI, Compix Inc., Cranberry, USA). The numbers of nuclei per fibre, corresponding to the number of attached cells, were counted using SigmaScan5 software. The average cell number was determined from n=18 and comparisons between sample groups were made using ANOVA and data are reported as mean ±standard deviation.

Assessment of cell confluency, spreading and patterning. To monitor cell growth, cell spreading and cell patterning, HUVEC-seeded fibres were kept in culture in an incubator (37° C., 5% CO₂) for 4 hours. The culture media were replenished every 2 days. For cell staining, cell-seeded fibres were gently washed with PBS (3 times) and fixed in a formaldehyde solution (3.75% wt/v) in PBS for 15 minutes. Following three washings with PBS, cells adhered on fibres were then permeabilized using a Triton X-100 solution (0.5% v/v in PBS) (Sigma Chemical Co.) for 5 minutes. Following two rinses in PBS, the samples were incubated in a mixture of TRITC-phalloidin (1:100 dilution, cat. #P1951, Sigma Chemical Co.) and SYTOX Green Nucleic Acid Stain (1 μM, cat. #S7020, Molecular Probes, OR, USA) or with Hoechst 33258 (1:100 dilution) in a blocking buffer solution (a solution containing bovine serum albumin (20% in PBS)) for 1 hour at room temperature and in the dark. The staining solution was removed and following washes with PBS (3 times), the preparations were then visualized on an Olympus Fluoview 300 confocal microscope and on an epifluorescence microscope (Nikon, Eclipse TE 2000-S). Images were captured using a COOLSNAP-Procf camera on Image-pro plus imaging software. The double-stain allowed to adequately distinguishing cell cytoskeleton (i.e., actin filaments) and nuclei. All experiments were replicated two or more times. At least four fibres were examined for each sample.

The detailed examination of the acting filament and focal adhesion structures was very difficult directly on PET fibres, because of the lack of transparency of these fibres. Therefore, borosilicate glass substrates were surface modified and sterilized under the same experimental conditions than those used to surface activate PET fibres with the aim to gather more information on the effect of the surface coatings on the actin filament and focal adhesion of the cells seeded on the different coatings.

Borosilicate glass substrates bearing the different surface coatings were placed in the wells of a 12-well culture plate. HUVECs were seeded onto these samples at a density of 10 000 cells/cm² and kept in culture, as for PET fibres, for 4 days. Afterwards, the samples were fixed and permeabilized by the same procedure described above and incubated 1 hour with a primary antibody (Monoclonal Anti-Vinculin, Sigma, cat.#V4505) at a dilution of 1:25 in the same blocking buffer described above. Samples were then washed three times with PBS for 5 minutes each and then incubated with the secondary antibody (1:250 dilution of Anti-Mouse IgG, Sigma, cat. #A2304) in the same blocking buffer for 1 hour. Samples were washed twice with PBS (5 min each) then mounted face-down onto glass microscope slides. Edges of the samples were sealed with nail polish and kept in dark until epifluorecence microscopic observation. All experiments were replicated two or more times. At least four fields were examined for each sample to assess the degree of stress fiber and focal adhesion formation on each substrate.

To observe cell growth on the GRGDS (concentrations of 0.5 mg/ml and 1 mg/ml)-coated fibres during a 10-day cell culture period, alive cells were stained with dil-acetylated LDL (cat. #BT-902, Biomedical Technologies Inc., Stoughton, Mass., USA) at day 2. Then, samples were observed with phase contrast or confocal microscope every day.

### Example IV

### Results Obtained with Biologically Active Pet Fibres

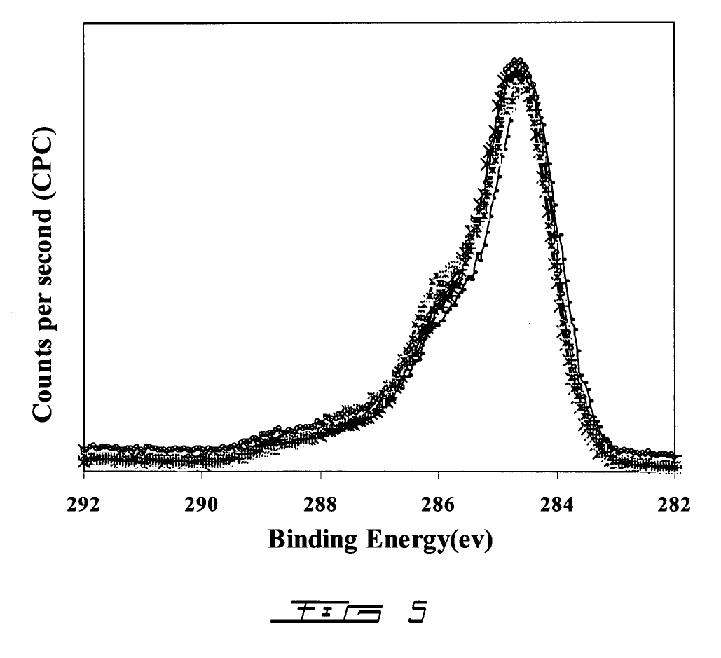

Surface analyses by XPS. As listed in Table 1, the successful surface modification of PET fibres by HApp, CMD, and GRGDS layers was immediately evident from the elemental composition determined by XPS analyses. XPS analyses of clean untreated PET fibres show C—C, C—O and C═O components in the C 1s high-resolution spectra (FIG. 4A) located at 285, 286.4 and 289 eV, respectively. This is in agreement with the PET fibre chemical composition and XPS analyses of PET fibres obtained from other studies.

XPS analysis of HApp layers on PET fibres (FIG. 4B and Table 1) indicated a polymer rich in hydrocarbon- and nitrogen-containing species, as observed previously on different substrates. The broad C 1s peak is associated with the variety of chemical structures, formed during plasma deposition from the HApp layer. As a result it is difficult to clearly resolve the C—N containing species from those containing C—O, which may result from the spontaneous quenching of carbon radicals within the film on exposure to air. The reduction in oxygen atomic concentration from 26.6% for an uncoated PET monofilament to 2.5% for a HApp-coated PET monofilament also indicates a uniform and thick plasma polymer layer. However, it is difficult to determine the thickness of the HApp layers based only on XPS analyses.

Surface modification by plasma polymerization has been applied extensively in the biomaterial field because the films produced are very smooth, pinhole and defect free, have good adhesive properties, can be deposited onto many different materials and complex geometries and generally contain low amounts of leachables. This method has been also shown to produce uniform film across silicon wafers and glass substrates. But, since plasma polymer films rapidly oxidize in air, they should be used immediately after preparation. The introduction of C—O (286.5 eV) and C═O (289 eV) observed in the high-resolution C 1s spectra indicated the successful grafting of carboxy-methyl-dextrans onto the HApp interlayer (as shown in FIG. 4C). A nitrogen signal was still evident, indicating that the CMD coating (in the dry state) is much thinner than the XPS analysis depth. These results are in agreement with those of an earlier study using the same multilayer structure.

To examine the effect of CMD molecular weight and CMD solution concentration during the immobilization procedure on the CMD coating, CMDs of 70 and 500 kDa molecular weights and two CMD solution concentrations (1 mg/ml and 2 mg/ml) were used. As depicted in FIG. 4, the XPS surface chemical compositions of the four CMD coatings were similar. As a result, CMD-coated fibres produced using CMD of 70 kDa molecular weight with a carboxylation ratio of 1:2 and a CMD solution concentration of 2 mg/ml were selected for cell testing of these fibres.

To justify the application of these fibres in cell and tissue culture applications, they would need to sustain standard sterilization procedures. As autoclaving is often the preferred method of sterilization for biomedical devices, the effect of autoclaving on fibre coatings was examined by XPS. XPS analyses revealed a small change in the surface elemental composition of the CMD-coated fibres (Table 1). Although we took good care to thoroughly rinse with 1M NaCl solution the CMD-coated fibres following CMD immobilization to remove physisorbed CMD molecules onto the fibres, it appears that some CMD detach during the harsh autoclaving conditions and/or that CMD coatings collapse during autoclaving. These hypotheses are supported by a decrease of the atomic O/C ratio following autoclaving CMD-coated fibres.

TABLE 1
Elemental Compositions of PET Fiber Surfaces Obtained by XPS Analyses

| | atomic concentrations (%) ± SD | | | atomic ratios (%) | | |
|---|---|---|---|---|---|---|
| samples | C 1s | O 1s | N 1s | N/C | O/C | exposure to oxyg [symbol] |
| PET fibers | 73.4 ± 2.1 | 26.6 ± 2.3 | 0.0 ± 0.0 | 0.000 | 0.36 | NA |
| PET + HApp | 88.1 ± 0.0 | 2.5 ± 0.6 | 8.7 ± 0.5 | 0.098 | 0.03 | 1 h |
| PET + HApp + CMD (1:2,* 70 kDa, 1 mg/mL) | 75.8 ± 1.8 | 18.2 ± 0.5 | 6.0 ± 1.3 | 0.079 | 0.24 | 3 days |
| PET + HApp + CMD (1:2, 70 kDa, 2 mg/mL) | 77.4 ± 0.6 | 17.8 ± 0.6 | 4.8 ± 0.7 | 0.062 | 0.23 | 3 days |
| PET + HApp + CMD (1:2, 70 kDa, 2 mg/mL) following autoclave | 80.1 ± 0.9 | 14.0 ± 1.5 | 5.4 ± 0.2 | 0.068 | 0.18 | 3 days |
| PET + HApp + CMD (1:2, 500 kDa, 1 mg/mL) | 77.2 ± 0.3 | 17.1 ± 0.6 | 5.7 ± 0.2 | 0.073 | 0.22 | 3 days |
| PET + HApp + CMD (1:2, 500 kDa, 2 mg/mL) | 77.1 ± 1.7 | 18.1 ± 0.1 | 6.0 ± 1.7 | 0.078 | 0.24 | 3 days |
| PET + HApp + CMD (1:2, 70 kDa, 2 mg/mL) + GRGDS (0.1 mg/mL) | 78.0 ± 3.5 | 16.3 ± 3.2 | 5.2 ± 0.4 | 0.067 | 0.22 | 5 days |
| PET + HApp + CMD (1:2, 70 kDa, 2 mg/mL) + GRGDS (0.5 mg/mL) | 78.2 ± 1.8 | 16 ± 1 | 5.9 ± 0.7 | 0.076 | 0.21 | 5 days |
| PET + HApp + CMD (1:2, 70 kDa, 2 mg/mL) + GRGDS (1 mg/mL) | 71.8 ± 2.0 | 21.8 ± 0.1 | 6.4 ± 0.6 | 0.089 | 0.30 | 5 days |

*1:2 means a carboxylation degree of 1:2.

Testing of surface-coated fibres towards endothelial cell behaviour. In multi-cellular organisms, cell-cell and cell-ECM interactions are mediated by cell adhesion receptors. Among these receptors, the integrin family comprises the most numerous and versatile group. Multiple integrin receptors with distinctive combination of α and β subunits have been identified on the surface of vascular endothelial cells. Among them, α5β1 and αVβ3, which bind to the ECM proteins fibronectin and vitronectin, respectively, are the best characterized and appear to be critical in the establishment and stabilization of endothelial cell monolayer. The adhesion of endothelial cells on glass or polymeric substrates is promoted by RGD immobilization on these surfaces. The RGD peptide sequence is found in many extracellular matrix proteins (e.g., fibronectin, vitronectin, collagen, and fibrinogen) and is the binding motif for cell attachment receptor (integrin αVβ1) present onto several cell membranes. The process of integrin-mediated cell adhesion comprises four different partly overlapping events: 1) cell attachment, 2) cell spreading, 3) actin cytoskeleton organization, and 4) focal adhesion formation. Firstly, in the initial attachment step, the cell makes contact with the surface and some ligand binding occur, allowing the cell to withstand gentle shear stresses. Secondly, the cell body begins to flatten and its plasma membrane spreads over the substrate. Thirdly, actin organizes into microfilament bundles, referred to as stress fibers. Fourthly, focal adhesion points form, which link the ECM to molecules of the actin cytoskeleton.

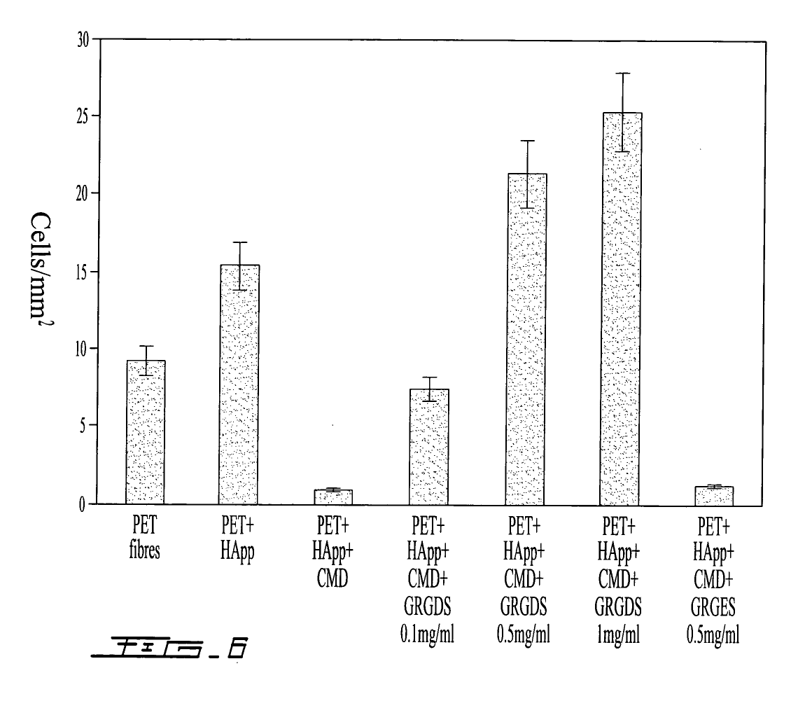

Endothelial cell attachment on surface-modified fibres. Cell adhesion and cell spreading assays were in good agreement with the expected results that were based on the chemical analysis and provided insight into how HUVECs respond to PET fibres covered by the different thin films. Cell adhesion was studied on all samples and expressed as cells/mm² of fibre surfaces. As expected, cell adhesion was reduced on CMD-coated surfaces (FIG. 5). In fact, HUVEC adhesion was the lowest on CMD-modified PET fibres, whereas HApp- and GRGDS-coated fibres promoted the highest cell adhesion (FIG. 5; HApp-coated fibres: 16±1 cells/mm² and GRGDS- or GRGES-bearing fibres (0.1 mg/ml GRGDS): 8.0±0.3 cells/mm², (0.5 mg/ml GRGDS): 21±4 cells/mm², (1 mg/ml GRGDS): 25±2 cells/mm²; (0.5 mg/ml GRGES): 1.0±0.2 cells/mm²-p<0.05).

The HApp coating is solid and dense, and therefore contributes minimal interfacial steric-entropic repulsion effects. It also possesses a low density of positive surface charges in pH 7.4-buffered saline solution. Hence, interfacial electrostatic forces are small, and protein adsorption onto the HApp layer may occur predominantly by dispersion and hydrophobic forces. Therefore, it suggests that cell adhesion on HApp-coated surfaces, in our study, is due to serum born protein adsorption. In the case of non-coated PET fibre, the small amount of cell adhesion may occur due to hydrophobic and dispersion forces as described above.

Surface immobilization of CMD on HApp-coated PET monofilaments significantly reduced HUVEC adhesion (FIG. 5; 1.00±0.04 cells/mm²; p<0.05). It has been reported that anionic carboxylic dextrans inhibit cell proliferation, perhaps due to an inability for the cells to adsorb onto these surfaces. Such surface coatings might be useful for the fabrication of coatings resistant to non-specific cell adhesion and cell colonization. A further advantage of CMD coatings is that they can be used as interlayers for the covalent immobilization of cell-adhesive glycoproteins or other specific biological signalling proteins. Such constructs enable the fabrication of systems that exquisitely possess specific biological responses since the non-adhesive CMD interlayer would limit and/or delay other non-specific undesired biological responses.

McLean et al.³ reported that the ability of CMD coatings to resist cell colonization significantly depends upon the mode of fabrication of the CMD coating. They reported that electrostatic interfacial forces are not the dominant factors governing the cell and tissue responses. However, a minimum CMD coating thickness or coverage is required for effective antifouling. Before interpreting biological responses of surface coatings, it is, however, essential to ascertain that such interpretations are not due to artifacts superimposed on the inherent interfacial properties of the coatings. For example, the presence of gaps in the surface coating could influence the results, but these would be difficult to find. As the HApp itself is cell adhesive, data presented in FIG. 5 could be interpreted as a result of incomplete CMD coverage and/or too thin CMD layer. The CMD coating grafted onto the HApp layer may therefore be seen as a “carpet pile” structure as described elsewhere³ in which polysaccharide chains are randomly attached by multiple pinning points and contain protruding loops and chain ends as well as “trains” (chain lengths that are pinned close to the surface). Loops and tail ends extend into the aqueous solution away from the solid surface because of their high solubility in water and the drive for charged groups to spread out away from the interface. Such protruding loops and chain ends provide steric-entropic hindrance to protein adsorption and cell attachment. The very low cell adhesion observed on CMD-coated fibres indicates that such steric-entropic repulsive interfacial forces were present. Yet, the small amount of cell adhesion observed indicates that the magnitude of the repulsive steric-entropic forces were not sufficient to completely overcome the attractive interfacial forces. But these coatings were capable to limit these non-specific adsorption events to allow the RGD surfaces to specifically control the cell responses.

Surface grafting of GRGDS peptides on CMD-coated PET monofilaments promoted cell adhesion at levels significantly higher than those observed on CMD-coated fibres. Also, cell adhesion significantly depended on the GRGDS solution concentration used during the grafting procedure (FIG. 5; GRGDS-bearing fibres (0.1 mg/ml GRGDS): 8.0±0.3 cells/mm², (0.5 mg/ml GRGDS): 21±4 cells/mm², (1 mg/ml GRGDS): 25±2 cells/mm²-p<0.05). Moreover, cell adhesion on the GRGES (inactive control, FIG. 5; 1.0±0.2 cells/mm²; p<0.05) was significantly lower than the amounts observed on GRGDS-bearing samples. These results suggest that the significant gains in endothelial cell adhesion on PET fibres bearing surface-grafted GRGDS were due to the biospecific responses of HUVEC surface integrins towards the GRGDS ligands available on the fibre surfaces. Thus, cell adhesion was predominantly modulated by the incorporation of adhesion-promoting components on the fibre surface rather than random adsorption of serum-borne cell adhesive proteins. The small amount of cell adhesion observed on non-specific surfaces bearing immobilized GRGES peptide sequences may be supported by the above hypotheses postulated to explain the very small amount of cell adhesion observed on CMD-coated fibres (i.e., incomplete coverage of HApp-coated surface by CMD layer). Also, GRGES-coated fibres might contribute to some electrostatic forces that would attract proteins and cells.

The small cell adhesion observed on GRGDS-coated fibres produced using the 0.1 mg/ml GRGDS solution could be explained by the presence of an insufficient surface density of RGD molecules. This is supported by a low surface concentration of nitrogen found in XPS analyses of these RGD coatings (i.e., 0.1 mg/ml). It is possible that the GRGDS molecules are much smaller than the extended chains of the CMD layer. In this case, CMD chains could act as a “cover” for the small GRGDS molecules available in such a low density. CMD loops and chain ends would provide steric-entropic hindrance for the cells to access the RGD molecules when available in such a low density. This is in agreement with another study in which reduced cell attachment was observed when the spacer moiety was too long. It was claimed that reduced cell attachment was not only because of the increasing entropy of the longer flexible spacer chains which opposes strong binding, but also because cells would prefer somehow a tight binding to more rigid surfaces. RGD peptides located at the end of flexible spacing moieties increased cell binding activity that may be caused by local enrichment of ligands. To obtain a reasonable number of adherent cells on the samples, a concentration of 0.5 mg/ml GRGDS was chosen for following experiments.

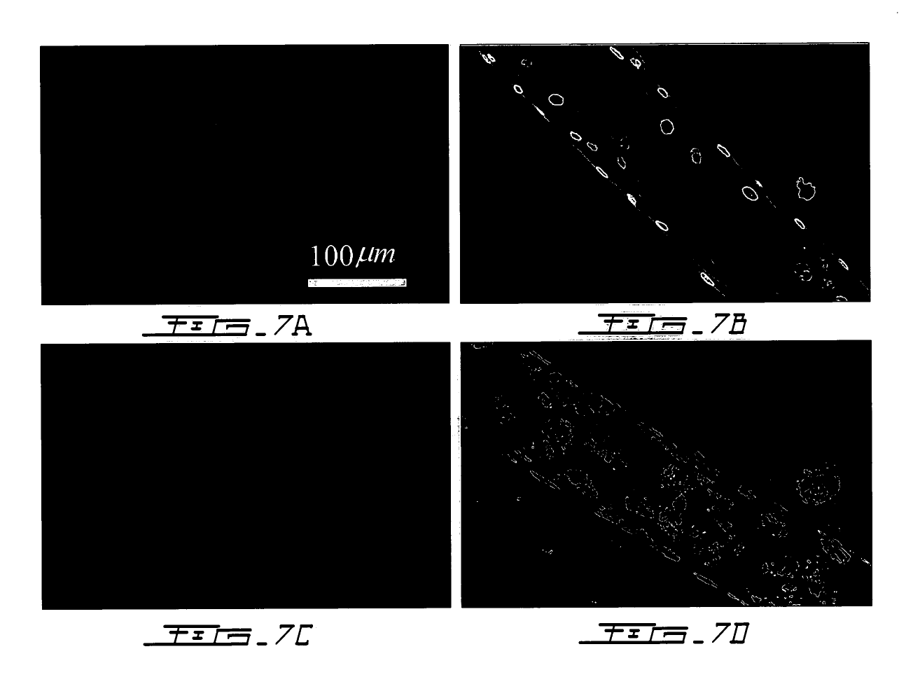

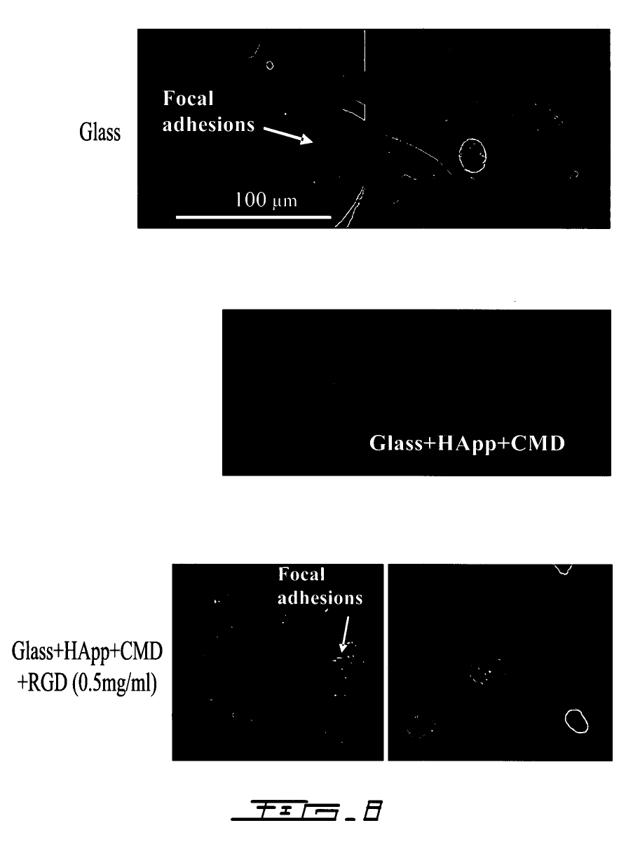

Effect of surface-modified fibres on endothelial cell confluency, spreading and patterning. Cell morphology is one of the criteria used to study cell adhesion events and cell spreading. To observe the effect of the fibre surface chemistry on HUVEC spreading, HUVECs were double-stained to identify cell cytoskeleton (i.e., actin filaments) and nuclei. According to the results obtained by day 4, it seems that endothelial cell adhesion, stability and spreading depend on the surface chemistry of the samples (see FIG. 6 and FIG. 7). FIG. 6 shows the degree of attachment and spreading of endothelial cells on surface-modified PET fibres at day 4. Despite of some cell adhesion on the CMD-coated (FIG. 6 1.00±0.04 cells/mm²) and GRGES-coated fibres (FIG. 6. 1.0±0.2 cells/mm²) at 4 hours, no cell remained on these surfaces by day 4 (FIGS. 7 and 7, respectively). It can be hypothesized that early cells deposited on these CMD surfaces can detach because of insufficient adhesion and spreading. In contrast, cells were confluent and largely spread on GRGDS-coated fibres (FIG. 7). These observations suggest that GRGDS-coated fibres facilitate cell adhesion, and cell spreading through their integrins towards the GRGDS ligands available on the fibre surfaces.

Dil-acetylated LDL staining. To clarify if GRGDS coating is stable under cell culture conditions for a longer period of time, HUVECs were seeded on the GRGDS-coated fibres (0.5 mg/ml GRGDS concentration) and maintained in culture for a period of 8 days. At day 2, cells were stained with dil-acetylated LDL and daily observation with phase contrast and confocal microscope showed the uniform cell distribution (FIG. 6D), growth and stability over the fibre surface.

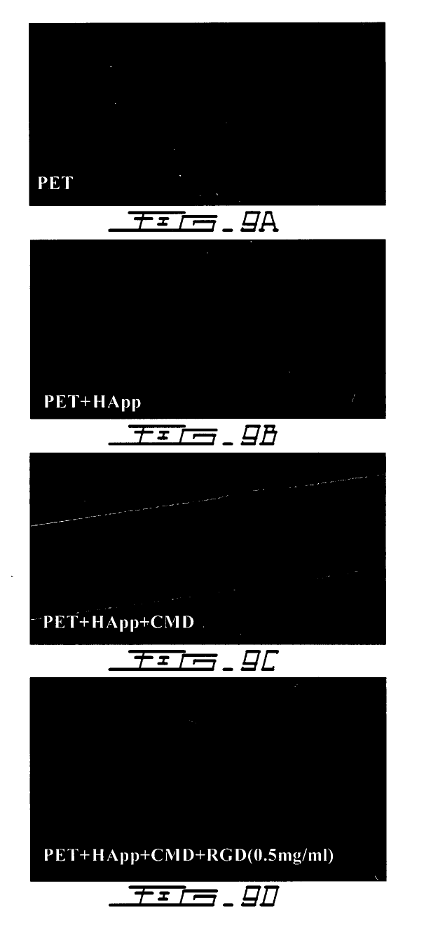

Actin stress fibres and focal adhesions. Few studies have investigated endothelial cell adhesion in terms of actin cystoskeleton and focal adhesions on the surface-coupled adhesive RGD peptide. As actin stress fibers and focal adhesions are critical for cell survival, HUVECs were seeded on uncoated, HApp+CMD- and HApp+CMD+RGD-coated glass substrates to study the effect of these coatings on actin stress fibers and focal adhesions. In FIG. 8, the red and green staining denotes actin stress fibers and focal adhesion points, respectively. As expected, there was no cell on CMD-coated surfaces to be observed for actin stress fibres and for focal adhesions. The sharp focal vinculin staining noted on RGD-coated substrates was absent on uncoated glass. Also, actin staining was intense on RGD-coated surfaces when compared to the uncoated glass (FIG. 8). This observation demonstrates the bioactivity of RGD-coated substrates. It also shows that the RGD peptides were strongly surface immobilized on the solid substrates, which is essential to promote strong cell adhesion, because formation of focal adhesions only occurs if the ligands can withstand cells contractile forces.

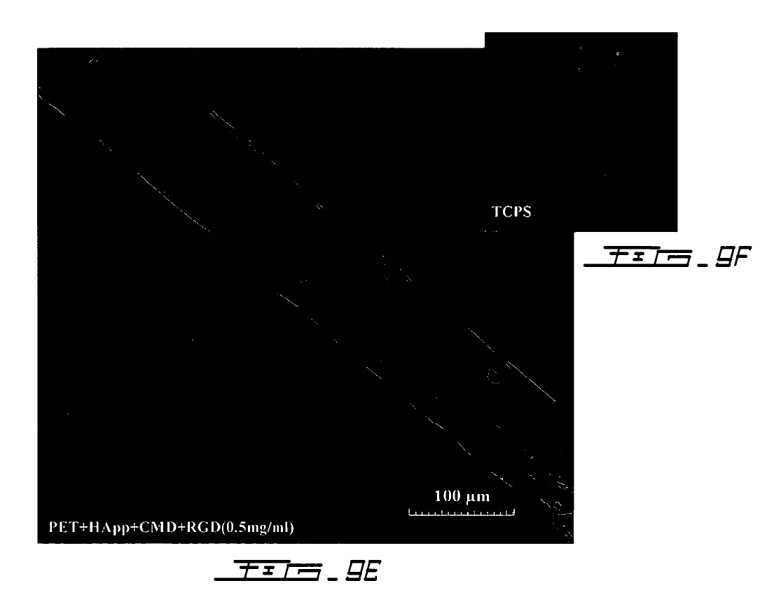

Cell elongation along the fibre axis. To observe the effect of the fibre curvature and fibre surface chemistry on HUVEC spreading and orientation in long-term cell culture (i.e., 10 days), HUVECs were double-stained with TRITC-phalloidin for cell cytoskeleton (i.e., actin filaments) and with Hoechst 33258 for nuclei. Epifluorecence microscope observation showed that, on untreated PET fibres and on HApp- and GRGDS-coated fibres, cell bodies were elongated along the fibre axis (FIGS. 9A, 9B, 9D) compared to those observed on the flat surfaces of the tissue culture polystyrene plates (FIG. 9F), which were randomly spread. Confocal microscope photographs show that actin filaments were oriented parallel to the fibre axis; FIG. 9E shows a typical image illustrating this effect. Cell orientation along the fibre can be influenced by fibre curvature as previously reported that most cell types are known to orient and often move rapidly along fibres in the 5-108 micrometer range. More investigation will be warranted to better understand how fibre curvature and fibre diameter can affect cell responses.

Conclusions. Bioactive polymer fibres that encourage and control directional biological responses were developed, characterized and validated. For this purpose, RGD peptide, a cell adhesive sequence, has been selected and immobilized onto the surface of PET fibres (monofilaments), using a plasma polymer layer and a low-fouling and non-adhesive carboxy-methyl-dextran interlayer to modulate endothelial cell patterning in a 3D environment. Bioactivated fibres could be used to modulate cell patterning when dispersed in a 3D environment.

X-ray photoelectron spectroscopy (XPS) analyses enabled detailed characterization of the multi-layer fabrication steps. Human umbilical vein endothelial cells (HUVEC) were used to investigate cell patterning (i.e., adhesion, growth, spreading, and orientation) as a function of the fibre surface properties. As expected, cell adhesion was reduced on CMD-coated fibres as HUVEC adhesion was the lowest on these fibres, whereas amine- and GRGDS-coated fibres promoted cell adhesion, growth and spreading. These low-fouling CMD surface coatings (and possibly other low-fouling coatings not tested in this study) on polymer fibres could lead to the development of well-defined surface modifications that allow for the precise control of directional cellular interactions at the tissue-biomaterial interface, and ultimately improved performance of long-term biomaterial implants. Moreover, cell adhesion on the GRGES-coated fibres (non-active peptide control) was significantly lower than that observed on GRGDS-coated fibres. These observations suggest that the significant gains in endothelial cell adhesion on PET fibres bearing surface-grafted GRGDS were due to the biospecific responses of cell surface integrins towards RGD ligands available on the fibre surfaces. Cell adhesion increased by increasing GRGDS concentration. Cells on RGD-coated substrates formed well-defined stress fibres and sharp spots of vinculin, typical of focal adhesions. However, further studies will have to be undertaken to investigate in more detail the effect of RGD peptide surface density on cell responses. In addition, fibre pattern promoted cell orientation along the fibre axis in comparison to flat surfaces, and this parameter were more effective in long-term cell culture.

### Example V

### Bioactive PTFE Fibres

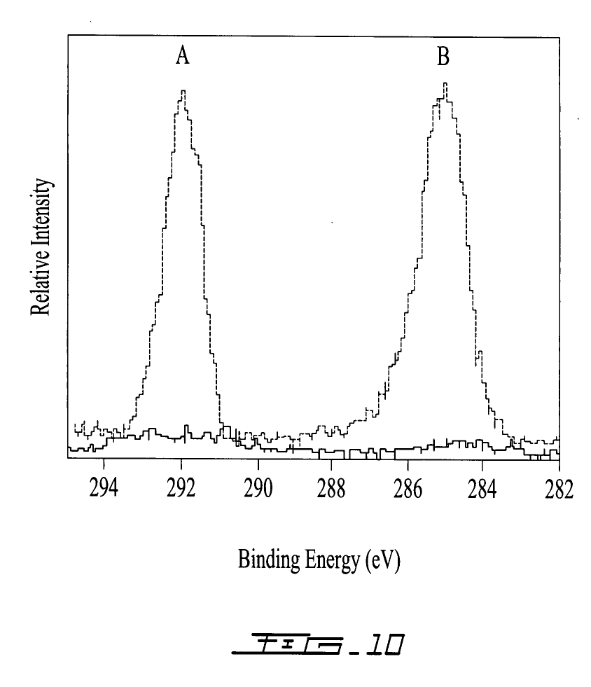

Hepthylamine plasma polymer (HApp) was deposited onto poly(tetrafluoroethylene) (PTFE, diameter of 200 μm) monofilaments according to Example II. XPS data showed the successful deposition of HApp onto PTFE monofilaments illustrated by a significant increase of the N/C ratio (0.09) indicating a high concentration of nitrogen containing functional groups on the surface. The results (Table 2 and FIG. 10) also showed complete attenuation of the fluorine signal indicating the presence of a coating thicker than 10 nm (the analysis depth of the XPS).

TABLE 2
Elemental compositions of polymer
surfaces obtained by XPS analyses.

| | Atomic concentrations (%) | | | | Atomic ratios (%) | |
|---|---|---|---|---|---|---|
| Samples | C 1s | O 1s | N 1s | F1s | N/C | O/C |
| PTFE | 23.46 | 0 | 0 | 76.54 | 0 | 0 |
| PTFE + Happ | 88.41 | 3.45 | 7.98 | 0 | 0.09 | 0.039 |

Endothelial cell attachment and confluency on surface-modified PTFE fibres. Carboxymethyl dextran (CMD) interlayer and RGD and RGE peptides were covalently grafted onto HApp-coated PTFE monofilaments in the way described above (Example I). Human umbilical vein endothelial cells (HUVECs) were seeded onto surface-coated PTFE monofilaments (refer to Example II).

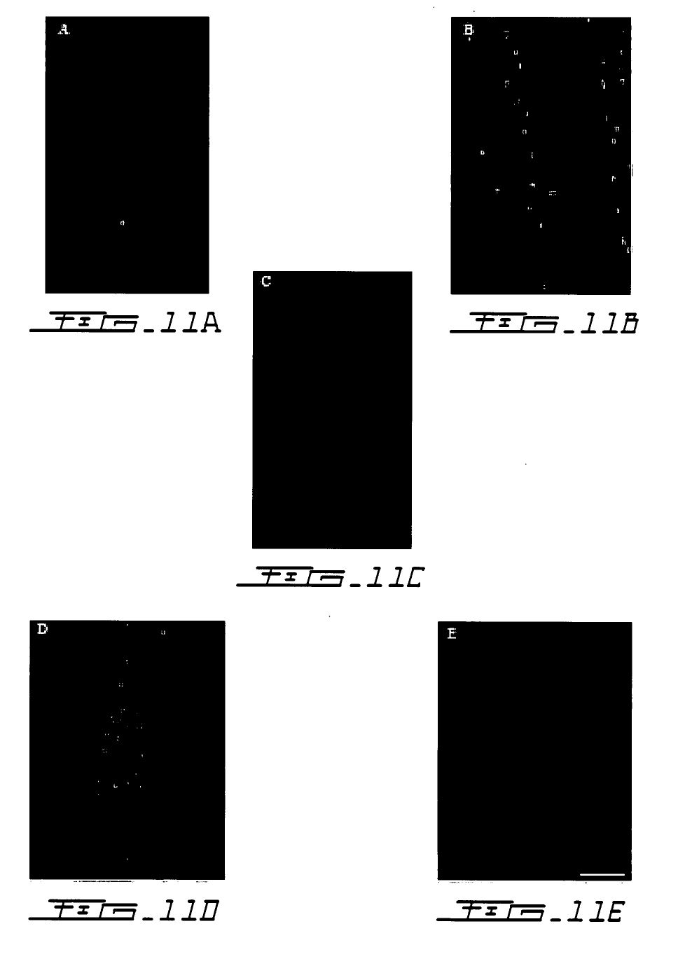

Cell adhesion was reduced on CMD-coated surfaces (FIG. 11C). In fact, ECs adhesion was the lowest on CMD-modified PET fibres, whereas HApp- and RGD-coated fibres promoted the highest cell adhesion and confluency (FIGS. 11B and 11D, respectively).

Surface grafting of RGDS peptides on CMD-coated PTFE monofilaments promoted cell adhesion at levels significantly higher than those observed on CMD-coated fibres. Moreover, cell adhesion on the RGE (inactive control, FIG. 11E) was significantly lower than the amounts observed on RGD immobilized samples. These results suggest that the significant gains in endothelial cell adhesion on PTFE fibres bearing surface-grafted RGDS were due to the biospecific responses of ECs surface integrins towards the RGD ligands available on the fibres surfaces. Without wishing to be bound to theory, cell adhesion might be predominantly modulated by the incorporation of adhesion-promoting components on the fibre surface rather than random adsorption of serum-borne cell adhesion proteins.

### Example VI

### Bioactive Pet Fibres Covered with an Acetaldehyde Plasma Layer and a PEI Spacer

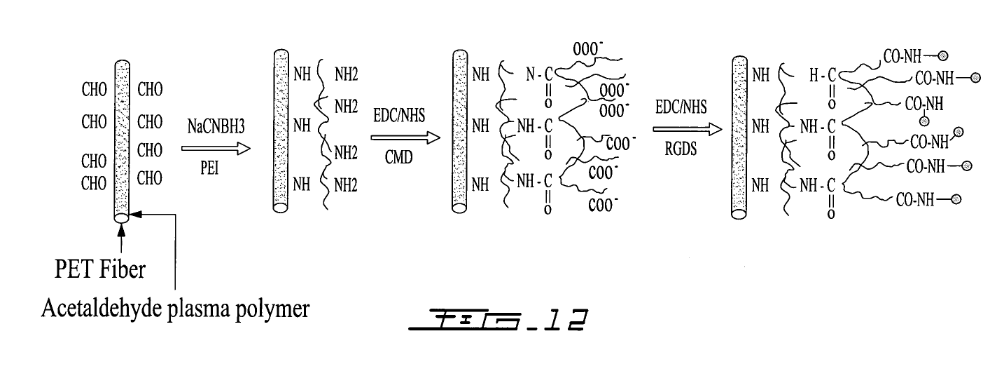

CMD was immobilized via a plasma step and poly(ethylenimine) (a polyamine spacer) by two solution reaction steps. Surface activation was undertaken by exposing the PET monofilaments to an acetaldehyde plasma in the same way as described for HApp in Example I and with the following operating conditions: Monomer pressure of 0.300 Torr, Power of 80 W, Frequency of 50 kHz, Deposition time of 70 s, and the distance between the electrode of 10 cm. The aldehyde groups formed on the surface were then reacted directly with a solution of polyamine (PEI, Polyscience Inc.) (PEI 3 mg/ml in Milli-Q water at pH 7.4) and an excess of sodium cyanoborohydride (3 mg/ml) added to act as a reducing agent. Amine groups provided by the polyamine-coupled fibres surfaces were then used to couple CMDs as described for HApp-coated fibres surfaces earlier. Afterwards, RGD molecules were immobilized onto the carboxylic groups of CMD-coupled fibres surfaces in a similar fashion as described in Example I (FIG. 12).

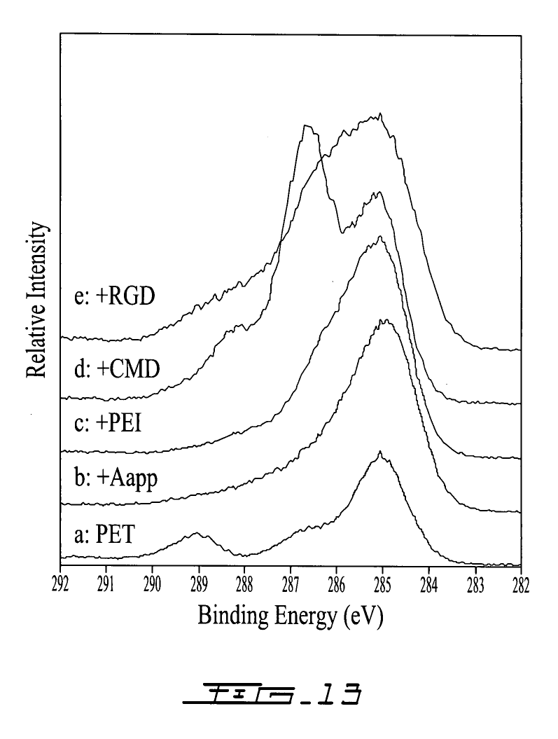

XPS analysis of Acetaldehyde plasma polymer (Aapp)-based coatings. For CMD immobilization via surface aldehyde groups and polyamine spacers, the acetaldehyde plasma polymer bonding layer and the polyamine spacer layer were analyzed by XPS. The decrease in oxygen atomic concentration from 26.6% in PET to 16.69% for Aapp-coated surface (Table 3) and the disappearance of shifts in 286.5 eV and 289 eV (FIG. 13B) show a thick plasma polymer coating. The XPS C 1s spectrum of PEI-grafted AApp is shown in FIG. 13C. The increase in nitrogen content (but not oxygen) shown in Table 3 suggests that the increase in height of the C 1s shoulder at 286.5 eV is attributed to the presence of C—N species of the PEI. Table 3 shows the successful attachment of CMD 1:2 to the acetaldehyde-PEI surface. The presence of C—O (286.5 eV), C═O (288.5 eV), observed in the high-resolution C1s spectra indicated the successful grafting of CMD onto the PEI interlayer (FIG. 13D). Looking at the N 1s signal from the PEI layer following CMD grafting, the presence of PEI at the outer surface of the coating is detected, possibly as a result of intercalation within the polysaccharide layer. Another possibility is that this result represents an artifact due to structural rearrangement of the coating during dehydration/evacuation prior to XPS analysis. The XPS C1s spectrum of CMD (FIG. 13D) indicate that the absolute amount of CMD bound on the surface is consistently significantly larger when the CMDs were immobilized via a polyamine spacer layer as compared with attachment directly onto the rigid, flat HApp surface (FIG. 13C).

TABLE 3
Elemental composition determined by XPS of the coatings

| | Atomic concentration (%) | | | | | | Atomic ratio | |
|---|---|---|---|---|---|---|---|---|
| Surface | C1s | O1s | N1s | S | Sl | Cl | N:C | O:C |
| PET | 73.4 | 26.6 | 0.0 | 0.0 | 0.0 | 0.0 | 0.0 | 0.36 |
| PET + Aapp | 80.14 | 16.69 | 3.17 | 0.0 | 0.0 | 0.0 | 039 | 0.20 |
| PET + | 76.10 | 12.80 | 9.65 | 0.62 | 0.83 | 0.0 | 0.13 | 0.17 |
| Aapp + | | | | | | | | |
| PEI | | | | | | | | |
| PET ++ | 66.50 | 25.97 | 6.03 | 0.0 | 1.50 | 0.0 | 0.090 | 0.39 |
| Aapp + | | | | | | | | |
| PEI + CMD | | | | | | | | |
| 1:2(70 kDa) | | | | | | | | |
| 1 mg/ml | | | | | | | | |
| PET + | 68.12 | 21.98 | 7.77 | 0.0 | 1.35 | 0.76 | 0.11 | 0.32 |
| Aapp + | | | | | | | | |
| PEI + | | | | | | | | |
| CMD + | | | | | | | | |
| RGD (0.1 | | | | | | | | |
| mg/ml) | | | | | | | | |

XPS analysis of samples containing grafted RGDS peptide on surface immobilized CMD is shown in FIG. 13E. A dominate 288.5 eV (C═O/N—C═O) component was observed. Other components were also observed, including 286.5.eV (O—C) and increase in nitrogen atomic concentration from 6.03% in CMD coated surface to 7.79% in RGD coated.

### Example VII

### Various Cell Types Cultured on Treated Bioactive Membranes

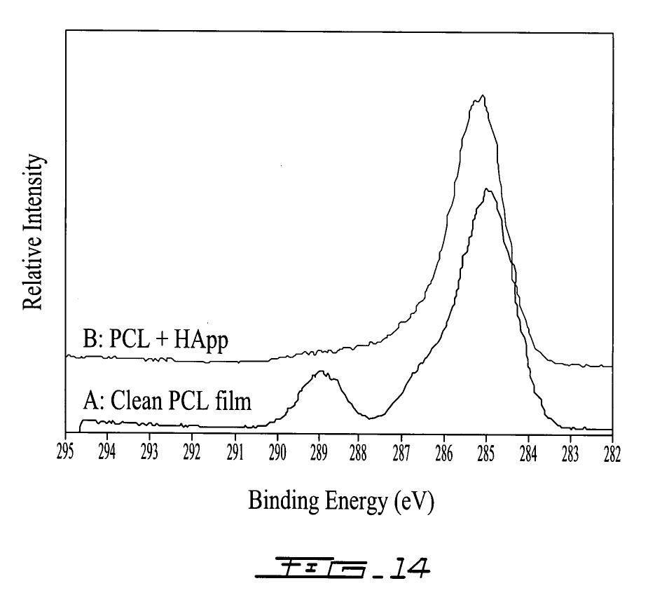

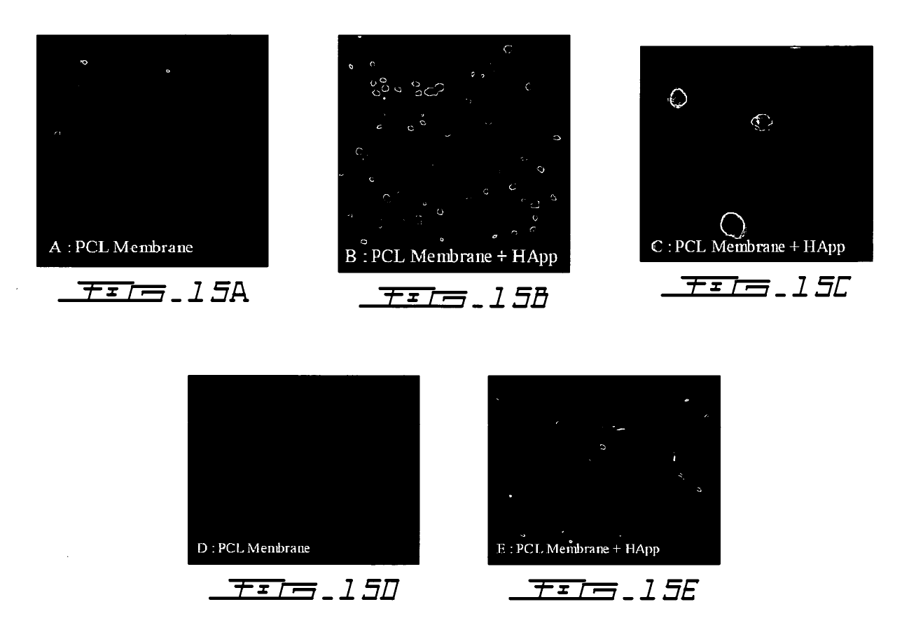

Biocompatible polymeric material, polymer fibres and membranes were surface-coated by using HApp plasma polymer (Example I). XPS data (Table 4 and FIG. 14B) showed the successful deposition of HApp on to the Poly(epsilon-caprolactone) (PCL) membrane by a significant increase in the N/C ratio (0.077) indicating a high concentration of nitrogen containing functional groups on the surface. Endothelial cells have been deposited and cultured on the membrane according to the details set forth in Example II. Fibroblasts have been cultured in a DMEM medium containing 10% fetal bovine serum and antibiotics until confluency. Cells have then been deposited on the membrane at a concentration of 1×10⁶. Treated bioactive PCL membranes promoted cell adhesion and spreading of endothelial cells and fibroblasts, thereby indicating that the treatment enabled the adhesion and spreading of various cell types. In FIG. 15, endothelial cells have been stained using the SYTOX™ fluorochrome and NBD-phallacidin for actin filaments whereas fibroblasts have been stained using a Live/Dead assay (Molecular Probes, Invitrogen).

TABLE 4
Elemental composition of polymer surfaces
obtained by XPS analyses.

| | Atomic concentrations (%) | | | Atomic ratios (%) | |
|---|---|---|---|---|---|
| Samples | C 1s | O 1s | N 1s | N/C | O/C |
| PCL membrane | 79 | 21 | 0 | 0 | 0.26 |
| PCL membrane + HApp | 85.3 | 8.1 | 6.6 | 0.077 | 0.095 |

### Example VIII

### Bioactive Fibres Having Different Diameters

Surface topography such as fibre diameter or its curvature is of particular interest to modulate the behaviour of cells such as proliferation, migration, and locomotion. Typical examples of the effect of fibre diameter or its curvature on the cell behaviour are the endothelial cell lining on polythylene terephthalate (PET, Dacron™) woven fibres for the artificial vascular grafts and orientation of fibroblast cells along the fibre axis (21-108 μm diameter fibre). The literature in this case is, however, far less, and little is known about it, moreover existing data is not clearly in agreement.

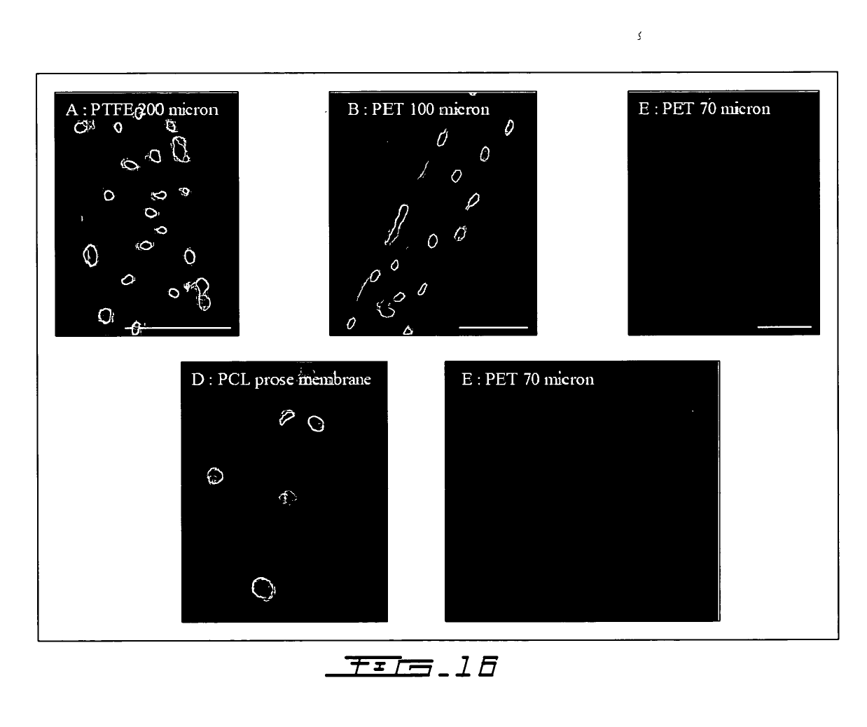

In this study, polymer fibres and membranes were surface-coated by using HApp plasma polymer (refer to Example I). Afterwards, surface-coated fibres were used to investigate the effect of fibre diameter on endothelial cell behaviour. FIG. 16 depicts the cell interaction on the surfaces of PTFE (200 μm diameter) and PET (70 and 100 μm diameters) fibres. Results indicated that on large curvature as over 200-micrometer diameter (FIG. 16A), the cells migrated easily in both directions (transverse and perpendicular) like a plain surface. On curvature with 100-micrometer diameter (FIG. 16B), cells tended to move onto plain surface rather than onto the concave side, resulting in that almost all cells were oriented perpendicular along the fibre direction. On fibre diameter about 70 micro-meter (FIG. 16C), cells tend to wind or wrap the fibre. FIG. 16D shows the cell body totally wrapped. For these reasons, this in vitro study provided the evidence that polymer fibre dimension can be used to selectively control cell functions.

### Example IX

### Different Biologically Active Molecule

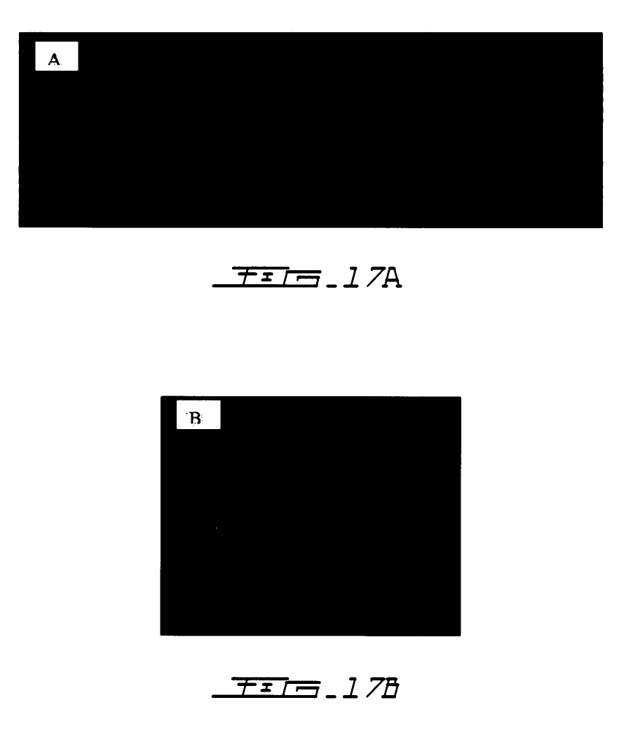

Gelatin (100 μg/ml) was immobilized onto CMD coated PET fibres in the same way as described for RGD peptide immobilization above (Example I) and human umbilical vein endothelial cells (HUVECs) were seeded onto surface coated PET monofilaments (and cultured as described in Example II). The results shown in FIG. 17 indicate that gelatin promoted endothelial cell adhesion and spreading onto the surface of 100-μm diameter poly(ethylene terephtalate).

### Example X

### Bioactive Fibres to Guide Angiogenesis (Micro-Vessel Formation)

Tissue engineering was originally developed as an alternative therapy for the treatment of tissue loss or end-stage organ failure, thereby resolving the shortage in the supply of tissues and organs for transplantation therapy. Although significant progress has been made in this field in recent years, the problem of retaining viable cells in thick complex tissue constructs in vitro (i.e., during culture) as well as in vivo (after implantation) has not been fully resolved, yet. Upon implantation, the tissue construct must be rapidly vascularized to sustain cell survival and later cell and tissue integration because the host's vascularization is often not sufficient to feed the implant. Therefore, the development of methods to pre-vascularize tissues is of critical importance to allow the culture of tissue mass with efficient nutrient supply and waste removal.

Angiogenesis i.e., the formation of neo-vasculature, was first described in vitro by Folkman and Haudenschild in the 80s. In fact, endothelial cells organize spontaneously into capillary-like structures with the presence of lumina. Since angiogenesis occurs in a 3-D environment, many cell culture systems have been developed in 3-D matrices to study angiogenesis. One interesting concept was the use of endothelial cell-seeded micro-carriers that were embedded in fibrin gel in which capillaries radiated in a “spoke-like” fashion to invade the fibrin gel in the presence of growth factors. Accordingly, in the present study, polymer fibres were used as a scaffolding material to produce a pre-vascularized construct with a directional micro-vessel network.

Most cell types are known to orient and can often move rapidly along fibres in the 5-108 μm diameter range, which has been referred to as “contact guidance”. Surface topography, as well as fibre curvature, is of particular interest to modulate the patterning of cells such as their proliferation and migration, as recently shown with human umbilical vein endothelial cells (HUVECs). Furthermore, pre-coating a biomaterial surface with a layer of a desired protein/peptide or other biologically active molecules can exert predictable biomedical responses.

Considering the above discussion, the present study also aims to develop bioactive fibres and to use them to control and guide micro-vessel network patterning.

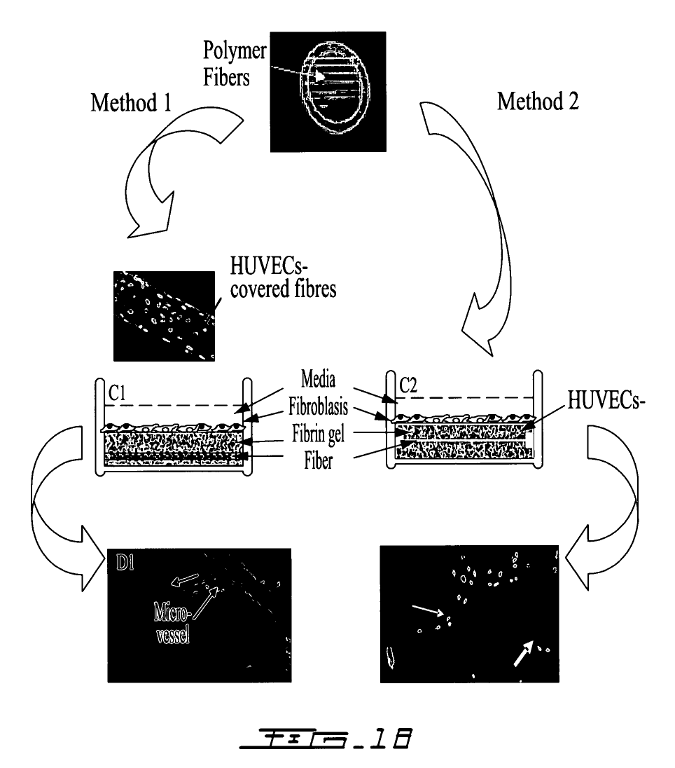

In this study, RGD peptides or gelatin were immobilized onto the surface of 100-μm diameter poly(ethylene terephthalate) (PET) monofilaments, via an n-heptylamine plasma polymer (HApp) bearing amine groups and a low-fouling carboxy-methyl-dextran (CMD) interlayers. Subsequently, two in vitro methods were designed with untreated and surface-modified polymer fibres embedded in fibrin gel to demonstrate the feasibility to induce directional micro-vessel formation within a 3-D environment (FIG. 18). The two systems consisted to introduce endothelial cells (i.e., HUVECs) either present on the surface of the fibres or by dispersing cells around cell-free fibres.

Methods

Materials. Commercially available, 100-μm diameter monofilaments, made of poly(ethylene terephthalate) (PET, ES305910, Good fellow, Devon, USA), were used. N-heptylamine (99% purity, 51958), used in the plasma polymerization, was obtained from Sigma-Aldrich (Saint-Louis, Mo., USA). Dextran (70 kDa molecular weight) was purchased from Amersham Bioscience (US14495, Upsala, Sweden). Dextran was carboxy-methylated to ca. 50%. GRGDS (44-0-23, Sunnyvale, USA) and GRGES (44-0-51, Sunnyvale, USA) peptides were purchased from American Peptide Company. Fibrinogen (F8630), thrombin (T3399), Aprotinin (A1153) were obtained from Sigma-Aldrich and bFGF (354060) from BD Biosciences (San Jose, Calif., USA).

Polymer Fibre Surface Modification

PET fibre preparation. To modify PET fibre surfaces, fibres were fixed onto non-conductive 3-mm thick Teflon holders of 2-cm diameter (FIG. 18A). These holders were designed for cell cultures in 12-multiwell plates to study micro-vessel formation. Fibres secured to the frames were cleaned by immersing them into a surfactant solution followed by thorough rinsing with a flow of Milli-Q water (of resistivity not less than 18.2 MΩ*cm) and finally blow-dried, using 0.2-μm filter-sterilized air flow.

Deposition of reactive amine groups onto PET fibres by plasma polymerization. PET monofilaments, being firmly fixed to the holders, were initially activated using Radio Frequency Glow Discharge plasma deposition to obtain a cross-linked, polymeric thin film bearing reactive surface groups onto which further layers could be immobilized by aqueous phase chemistry. Deposition of the thin plasma polymer films was performed utilizing vapors of n-heptylamine.

Carboxy-methyl-dextran (CMD) immobilization onto plasma-modified PET fibres. CMD was attached directly to n-heptylamine plasma polymer (HApp) layers using water soluble carbodiimide chemistry. For this purpose, 2 mg/ml CMD solutions were prepared in water. Once CMD was dissolved, 19.2 mg/ml of 1-ethyl-3-(3-dimethylaminopropyl) carbodiimide (EDC) and 11.5 mg/ml of N-hydroxysuccinimide (NHS) were added to the CMD solutions. Holders bearing the HApp-coated monofilaments were then immersed in the resultant activated CMD solution and left to react overnight at room temperature under vigorous agitation. To remove any non-covalently attached CMD molecules, treated fibres were rinsed for 24 hours under vigorous agitation in a 1 M NaCl solution, followed by a 24 hour immersion in water under agitation at room temperature before a final rinsing in water.

RGD peptide and gelatin grafting onto CMD-coated fibres. RGD immobilization was performed according to a sequential procedure. Autoclaved CMD-coated fibres were immersed in filter-sterilized (through low-binding 0.22-μm syringe filters, Millipore Corporation, Bedford, Mass., USA) solutions of EDC (19.2 mg/ml) and NHS (11.5 mg/ml) in PBS (1×, pH 7.4) at room temperature for 4 hours, followed by rinsing with PBS (pH 7.4; 2×15 min). Activated CMD-coated fibres were then incubated in a filter-sterilized (0.22-μm) peptide solution of 0.5 mg/ml RGD made in PBS (pH 8) for 2-3 hours at room temperature. After rinsing with PBS (pH 8, 3×15 min) to remove any unbound free peptide molecules, fibres were left to dry under a sterile laminar flow hood. RGE (0.5 mg/ml) and gelatin (100 μg/ml) were immobilized by the same procedure as described above.

Cell culture. Human umbilical vein endothelial cells (HUVECs) were purchased from Cambrex (cc-2519, Walkersville, Md. USA). HUVECs were cultured at 37° C. and 5% CO₂ in M199 culture medium (M5017, Sigma-Aldrich) containing 2.2 mg/ml sodium bicarbonate (Fisher, Fair Lawn, USA), 90 μg/ml sodium heparin (H1027, Sigma-Aldrich), 100 U/100 μg/ml penicillin/streptomycin (15140-122, Invitrogen Corporation, NY, Grand Island, USA), 10% fetal bovine serum (FBS, F1051, Sigma-Aldrich) 2 mM L-glutamine (25030149, Invitrogen Corporation, NY, Grand Island, USA), and 25 μg/ml endothelial growth factor supplement (ECGS, 356006, BD Biosciences, San Jose, Calif., USA). HUVECs between passages 2 and 4 were used in all experiments. Human fibroblasts extracted from human foreskin were cultured in M199 supplemented with 5% FBS at 37° C. and 5% CO₂. Fibroblasts between passages 3 and 5 were used in all experiments.

Method 1: HUVECs-Coated Bioactive Fibres Embedded in Fibrin Gel

Preparation of Fibres Covered by HUVECs. Cell Cultures on Pet Fibres Bearing different coatings were performed. Three specimens of cleaned, uncoated PET monofilaments (control) and of PET monofilaments coated with 1) HApp, 2) HApp+CMD+RGD and 3) HApp+CMD+Gelatin were investigated. Holders supporting the fibres were placed in the wells of 12-well tissue culture plates, then rinsed in sterile PBS (pH 7.4), and incubated in M199 culture medium for 1-2 hours to keep the fibres hydrated until cells were seeded. Then, 1 ml of M199 culture medium, containing 1×10⁶ cells (between passages 2 and 4), were directly poured into the well containing the fibre holder. Fibres were incubated in a CO₂ incubator at 37° C. and 5% CO₂. HUVECs were allowed to attach to the cell-adhesive fibres by incubating cells with fibres in M199 supplemented as indicated herein up to confluence. When the entire surfaces of the fibres were covered with HUVECs, they were used for the angiogenesis assays. The procedure is summarized in FIG. 18. It should be noted that cell confluence on uncoated PET and surface-coated PET fibres will not be achieved at the same period of time, because initial cell attachment on uncoated PET fibres in comparison to that on surface-coated fibres was too small, as shown in a previous study.

Micro-vessel network formation using HUVEC-covered cell adhesive fibres in a fibrin gel. Holders supporting HUVEC-pre-covered fibres were embedded in a fibrin gel prepared as follows. Holders with the HUVEC-covered fibres were placed in 12-multiwell tissue culture plates, then 1 ml of a 2.5 mg/ml fibrinogen solution in M199 culture medium (pH 7.4) with or without 0.15 Units/ml of aprotinin was added to each well, and finally 1 Unit of thrombin was added to each well. The fibrinogen solution was allowed to clot for 5 min at room temperature and then, at 37° C. and 5% CO₂ for 20 min. One milliliter of EGM-2 (Cambrex, cc-3162) (containing 2% FBS) was added to each well and 4×10⁴ fibroblasts were seeded on top of the gel. Culture medium was changed daily.

The culture system was monitored for 30 days. In these experiments, VEGF that is normally part of the EGM-2 formulation was omitted.

Method 2: Cell-Free Surface-Modified Fibres Sandwiched Between Fibrin Gels Containing HUVECs

In this system, uncoated cleaned PET monofilaments and PET monofilaments coated with 1) HApp, 2) HApp+CMD, 3) HApp+CMD+RGD, 4) HApp+CMD+Gelatin, and 5) HApp+CMD+RGE were investigated towards micro-vessel formation. Holders supporting the fibres were placed in the wells of 12-multiwell plates, then rinsed in sterile PBS (pH 7.4), and incubated in M199 culture medium for 1-2 hours to keep the fibres hydrated until cells were seeded. To make the underlying fibrin gel, 500 μl of fibrinogen solution (2.5 mg/ml) in M199 medium was added to each well of a 12-well culture plate and thrombin (1 U/ml) was added. After “polymerization” of fibrinogen into a fibrin gel (5 min at room temperature and 20 min at 37° C. and 5% CO₂), holders supporting the fibres were transferred onto the top of the gel and HUVECs suspended in serum-free M199 were seeded at a density of 1×10⁵ cells/well on the underlying fibrin gel. One milliliter M199 supplemented as indicated herein, was added to each well. After the formation of a confluent monolayer (˜24 h after seeding), the culture medium was aspirated and the same procedure was used again to generate a second fibrin gel layer over the cells. After fibrin gel polymerization, 1 ml of M199 supplemented with 0.15 U/ml aprotinin was added to each well.

Visualization of micro-vessel networks. To observe endothelial cell behaviour during the 30-day cell culture period, specimens were daily observed under phase contrast or confocal microscopy. In addition, cells were stained with Dil-Ac-LDL (BT-902, Biomedical Technologies Inc., Stoughton, Mass., USA) at day 3 and formation of tube-like structures (i.e., capillaries) was periodically traced. Images were recorded through a digital CCD camera (Regita 1300R, QIMAGING, Burnaby, Canada) and imaging software (SimplePCI, Compix Inc., Cranberry, USA). The systems were also visualized through an Olympus Fluoview 300 confocal microscope and the images were captured using a COOLSNAP-Procf camera on Image-pro Plus imaging software.

Images and processing. Fibres were imaged at low magnification (×4 and ×10) and recorded as high-resolution files (*.tif) to identify the effects of fibre-to-fibre distance over micro-vessel formation. Image-processing software (Scion Image; Scion, Frederick, Md.) and Photoshop were used to improve image quality; these images were used to quantify the number of vessel sprouts (sprouts>100 μm in length), which originated from the newly formed capillary situated along the fibre axis. The number of vessel sprouts was reported per unit of fibre length.

Statistical analysis. The average vessel number per unit of fibre length was calculated from n=9 and comparisons were made between groups using ANOVA. The data are reported as mean values ±standard deviation. The level of significance for change was set at a p value<0.05.

Histological sections. Fibrin gels containing fibre holders were fixed in 10% neutral buffered formalin for overnight. Constructs were gently removed from their wells, and proceeded to paraffin sections. Six-μm-thick sections were then cut and immuno-histochemisty double-stained using anti von Willebrand antibody (Factor 8, brown) and hematoxylin- and eosin as counter-stain for nuclear staining (blue).

Results and Discussion

Method 1: HUVECs-Coated Bioactive Fibres Embedded in Fibrin Gel

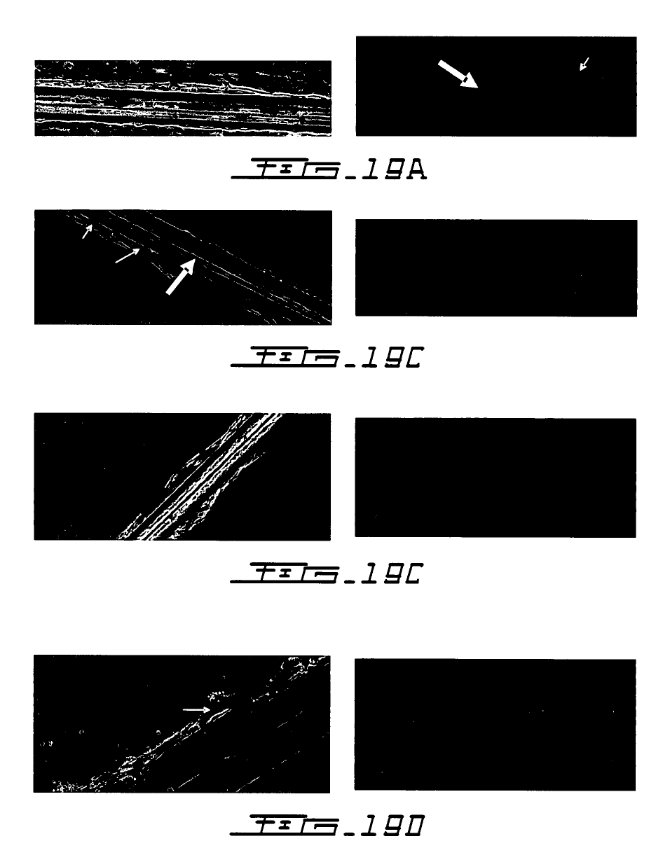

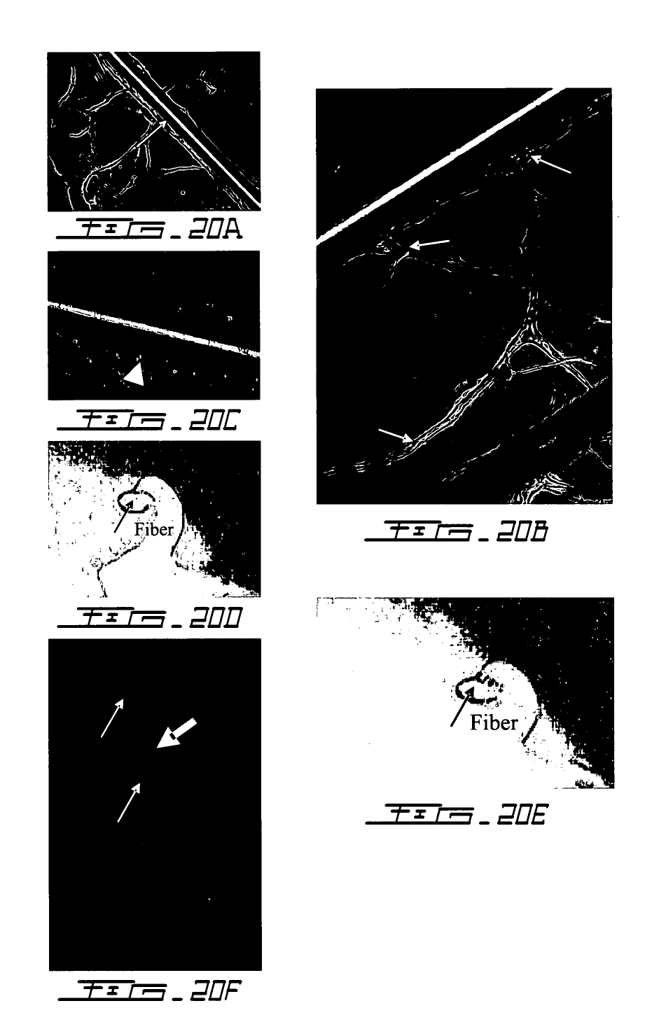

Within 2-3 days of cultures, elongated endothelial cell strands were formed along fibres axis. Sprouting was also observed from these cell strands (FIG. 19). Following 8-to-10 day culture, fully formed capillaries were generated, reaching lengths ranging between 0.5 to 1 mm. When left in the culture for up to 20 days, the capillary network continued to remodel itself, developing a network both along and between the neighboring fibres (FIG. 20A-20B). Micro-vessel numbers also increased as time increased (FIG. 20G). Phase-contrast microscopy images revealed the presence of a clear lumen within the vessels (FIGS. 20B and 20F). As additional evidence of lumen formation, histological sections of the samples showed the presence of lumens having multi-cellular architecture (FIG. 20D-20E). Conversely, endothelial cells on fibres were rather dispersed in the fibrin and did not exhibit any well-formed micro-vessel-like structures in the absence of a fibroblast monolayer (FIG. 20C).

Effect of fibre surface chemistry on angiogenesis stimulation. It was thought that by coating polymer fibres with bioactive molecules, it would be possible to optimize directional biological responses and, as a result, to develop directional micro-vessel network within a 3-D environment. In fact, the presence of bioactive molecules such as RGD on fibre surfaces significantly enhanced cell adhesion, as previously demonstrated. Surprisingly, all surface-modified fibres i.e., poly(ethylene terephthalate) (PET) fibres coated with n-heptylamine plasma polymer (HApp) layers, RGD and gelatin showed similar behaviours towards micro-vessel formation along fibre axis. RGD-coated fibres exhibited a “bridge-like” structure on the surface of the fibre (FIG. 19D), which subsequently formed short-length micro-vessels and branches by day 8-10 rather than elongated and separate micro-vessels. Since RGD peptides facilitate cell adhesion on these fibres, RGD-coated fibres may also attract more cells than non-RGD-coated fibres. Subsequently, these phenomena suggest that, afterwards, spread cells may detach from the fibres in some areas, giving raise to short-length micro-vessels with branching. Whereas cells that remain attached to the fibres may be activated to sprout with subsequent branching. Because endothelial cell adhesion and migration are essential components of micro-vascular development, the RGD sequence therefore regulates the interactions between sprouting endothelial cells and the extracellular matrix environment during angiogenesis.

Physical effect of fibre on angiogenesis stimulation. Cell shape, cell orientation, and cell migration can be guided by the topography of the substrate. This process, referred to as “contact guidance” or “topographic guidance” was clearly demonstrated by the alignment of cells with micro-machined grooves on solid substrates, as well as with fibres. At the molecular level, the response to the substrate topography may involve a mechanism similar to that for mechanical sensing. Cell movement can be guided by purely physical interactions at the cell-substrate interface. The direction of cell movement can be guided by manipulating mechanical strain within the flexible substrate. This suggests that, in the present study, endothelial cells located at the interface between rigid fibres and the soft fibrin gel were responding to two distinct substrate stiffness or substrate deformability. Subsequently, endothelial cells may follow a preferred growth direction along fibre axis.

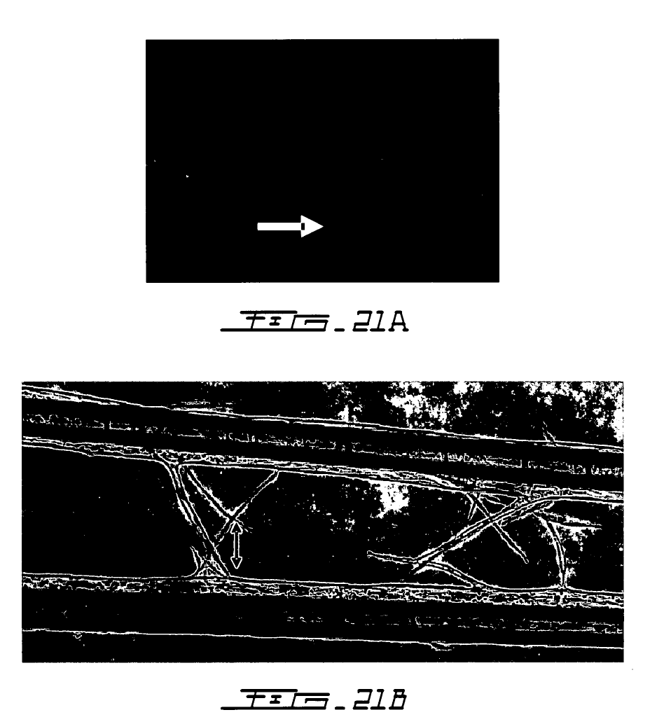

Effect of fibre-fibre distance on angiogenesis. The distance between two fibres is an important factor to develop an efficient micro-vascular network. Although the design of our fibre holder is limited to investigate only certain fibre-to-fibre distances (i.e., between ca. 0.1 and 2 mm), micro-vessel formation was mainly observed with fibres spaced between 200 and 600 μm (FIG. 21). Fibres distanced by less than 200 μm frequently showed juxtaposed micro-vessels along the respective fibres.

Method 2: Cell-Free Surface-Modified Fibres Sandwiched Between Fibrin Gels with HUVECs

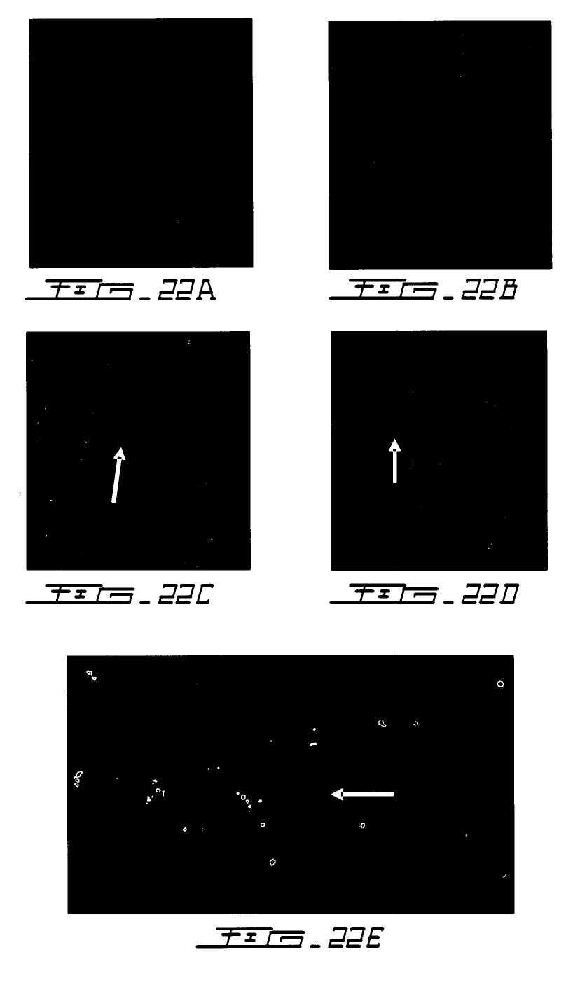

HUVECs and the holder supporting fibres were sandwiched between fibrin gels to investigate the potential of the fibres to orient micro-vessels as observed in Method 1. Non-coated, HApp-, RGD-, and gelatin-coated fibres as well as non-cell-adhesive fibres i.e., CMD- and RGE-coated fibres were shown to induce micro-vessel formation by day 4 of culture. The use of a fibroblast monolayer on the top of the gel was essential to achieve well developed micro-vessel networks (FIG. 22). Moreover, micro-vessels appeared to be more extensive than those observed in Method 1, in which fibres were pre-coated with HUVECs. Tubes were formed along fibres axis for all of the fibres tested. Branches and connections between micro-vessels were also observed. The presence of a monolayer of fibroblasts facilitates the endothelial differentiation, which was not the case in another study that uses a similar system. On the other hand, our finding is in good agreement with others that use beads.

### CONCLUSIONS

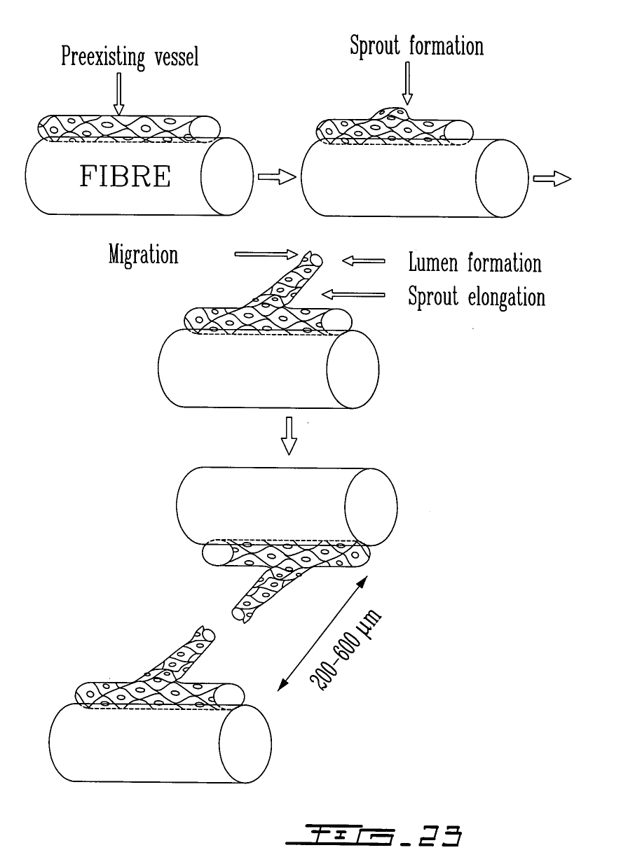

The objective of this study was to develop bioactive fibres and methods that use these fibres to guide micro-vessel formation in a given direction with the aim to engineer efficient pre-vascularization methods of tissue constructs. By using 100-μm diameter polymer fibres, the whole angiogenic process can be induced in vitro, ranging from endothelial cell activation to tube formation, through connections between branches (FIG. 23). Two in vitro methods were designed by using either untreated or surface-modified polymer fibres embedded in fibrin gel. These methods were successfully used to induce directional micro-vessel formation within a 3-D environment. The two systems consisted to introduce endothelial cells (i.e., HUVECs) either present on the surface of fibres or by dispersing cells around cell-free fibres. In both methods, micro-vessel formation occurred along fibres axis. A network was formed in which micro-vessels connected to each other from one fibre to another with an optimal fibre-to-fibre distance ranging between 200 to 600 μm. The presence of fibroblasts over fibrin gel promoted the formation and stabilization of the micro-vessels. The physical effect of fibres on the induction of angiogenesis was more important than that of fibres surface chemistry. By increasing culture time, micro-vessel number increased. Thus, the use of fibres is a promising approach to engineer tissue substitutes in which pre-vascularization is a pre-requisite to allow efficient nutrient supply and waste removal through the use of micro-vessel networks.

### REFERENCES

1. K. M. McLean, S. L. McArthur, R. C. Chatelier, P. Kingshott and H. J. Griesser, Hybrid biomaterials: Surface-MALDI mass spectrometry analysis of covalent binding versus physisorption of proteins. Colloids and Surfaces B: Biointerfaces 17 (1)(2000), pp. 23-35.
2. S. L. McArthur, K. M. McLean, P. Kingshott, H. A. W. John, R. C. Chatelier and H. J. Griesser, Effect of polysaccharide structure on protein adsorption. Colloids and Surfaces B:Biointerfaces 17 (1) (2000), pp. 37-48.
3. K. M. McLean, G. Johnson, R. C. Chatelier, G. J. Beumer, J. G. Steele and H. J. Griesser, Method of immobilization of carboxymethyl-dextran affects resistance to tissue and cell colonization. Colloids and Surfaces B: Biointerfaces 18 (3-4)(2000), pp. 221-234.
4. J. Y. Wong, J. B. Leach and X. Q. Brown, Balance of chemistry, topography, and mechanics at the cell-biomaterial interface: issues and challenges for assessing the role of substrate mechanics on cell response. Surface Science, 570 (1-2)(2004), pp. 119-133.
5. Curtis and M. Riehle, Tissue engineering: the biophysical background. Physics in medicine and biology 46 (2001), pp. 47-65.
6. R. Barbucci, S. Lamponi, A. Magnamb, D. Pasq, Micropatterned surfaces for the control of endothelial cell behaviour. Biomolecular Engineering 19 (2002), pp. 161-170.
7. Kh. G. Lee, S. J. Lee, J. H. Lee, H. Bang, Interaction of fibroblast cells onto fibres with different diameter. Korea Polymer Journal 7 (2)(1999), pp. 102-107.
8. D. C. Miller, A. Thapa, K. M. Haberstroh, and T. J. Webster, An In Vitro Study of Nano-fibre polymers for Guided Vascular Regeneration. Materials Research Society Symposium—Proceedings, 711 (2002), pp. 201-204.
9. Y. Zhu, Ch. Gao, Y. Liu, J. Shen, Endothelial cell functions in vitro cultured on poly (L-lactic acid) membranes modified with different methods. Biomed. Mater. Res. 69A (2004), pp. 436-443.
10. G. Maheshwari, G. Brown, D A. Lauffenburger, A. Wells, L G. Griffith, Cell adhesion and motility depend on nanoscale RGD clustering. J Cell Sci. 113 (Pt 10) (2000), pp. 1677-86.
11. U. Hersel, C. Dahmen and H. Kessler, RGD modified polymers: biomaterials for stimulated cell adhesion and beyond. Biomaterials, 24 (24)(2003), pp. 4385-4415.
12. Z. Miqin, T. Desai, and M. Ferrari, Proteins and cells on PEG immobilized silicon surfaces biomaterials. Colloids and Surfaces B: Biointerfaces 19 (10)(1998), pp. 953-960.
13. Y. Martin, D. Boutin, P. Vermette, Study of the effect of process parameters for n-heptylamine plasma polymerization on final layer properties. Thin Solid Films 515 (2007), pp. 6844-6852.

What is claimed is:

1. A bioactive composition comprising an arcuoid substrate and a polymeric film linked to the surface of the arcuoid substrate, wherein said bioactive composition controls the patterning of a cell.

2. The bioactive composition of claim 1, wherein the polymeric material is at least one of poly(ethylene terephtalate) (PET), poly(tetrafluoroethylene) (PTFE), poly(epsilon-caprolactone) (PLC), polyethylene, polypropylene, silicone, polyvinyl chloride, polyurethane, polymethyl(methacrylate), ultra-high molecular weight polyethylene, poly(glycolic acid), poly(lactic acid), polystyrene, nylon, polycarbonate and polysulfone.

3. The bioactive composition of claim 1, wherein the arcuoid substrate is a fibre.

4. The bioactive composition of claim 3, wherein the diameter of the fibre is between about 1 μm to about 1 mm.

5. The bioactive composition of claim 3, wherein the diameter of the fibre is between about 50 μm to about 250 μm.

6. The bioactive composition of claim 1, wherein the polymeric film is a cross-linked polymeric film.

7. The bioactive composition of claim 6, wherein the cross-linked polymeric film comprises at least one of a n-heptylamine monomer and an acetyldehyde monomer.

8. The bioactive composition of claim 1, wherein the polymeric film is a deposited polymeric film.

9. The bioactive composition of claim 1, wherein the polymeric film is cross-linked to the surface of the arcuoid substrate.

10. The bioactive composition of claim 1, wherein the polymeric film comprises a reactive group.

11. The bioactive composition of claim 1, wherein the thickness of the polymeric film is between about 2 nm to about 50 nm.

12. The bioactive composition of claim 1, further comprising a biologically active entity linked to the polymeric film.

13. The bioactive composition of claim 13, wherein the biologically active entity is coupled to the polymeric film by an amide bond.

14. The bioactive composition of claim 12, wherein the biologically active entity comprises a peptidic sequence.

15. The bioactive composition of claim 14, wherein the biologically active entity is at least one of a matrix protein, a portion of a matrix protein, a variant of a matrix protein, a denatured matrix protein, an enzyme and a growth factor.

16. The bioactive composition of claim 12, wherein the biologically active entity is at least one of a RGD peptide and gelatin.

17. The bioactive composition of claim 12, further comprising a spacer between the polymeric film and the biologically active entity, the spacer being linked to the polymeric film and to the biologically active entity.

18. The bioactive composition of claim 17, wherein the spacer is linked to the polymeric film and to the biologically active entity by an amide bond.

19. The bioactive composition of claim 17, wherein the spacer comprises at least one of a carboxymethyl dextran, a poly(ethylene oxide), a partially amino-functionalized dextran and heparin.

20. The bioactive composition of claim 17, wherein the spacer comprises a carboxymethyl dextran.

21. The bioactive composition of claim 20, wherein the molecular weight of the carboxymethyl dextran is between about 50 kDa to about 250 kDa.

22. The bioactive composition of claim 20, wherein the molecular weight of the carboxymethyl dextran is about 70 kDa.

23. The bioactive composition of claim 20, wherein the ratio of carboxylation of the carboxymethyl dextran is about 1:2.

24. A method of controlling the patterning of a cell, said method comprising culturing cells on the bioactive composition of claim 1, thereby controlling the patterning of the cell.

25. A method of modulating the cell patterning properties of an arcuoid substrate, said method comprising linking a polymeric film to the arcuoid substrate, thereby modulating the cell patterning properties of the arcuoid substrate.

26. A method of producing a bioactive composition capable of controlling the patterning of a cell, said method comprising linking a polymeric material on the surface of an arcuoid substrate, thereby producing the bioactive composition.

27. The method of claim 26, wherein the polymeric material is at least one of poly(ethylene terephtalate) (PET), poly(tetrafluoroethylene) (PTFE), poly(epsilon-caprolactone) (PLC), polyethylene, polypropylene, silicone, polyvinyl chloride, polyurethane, polymethyl(methacrylate), ultra-high molecular weight polyethylene, poly(glycolic acid), poly(lactic acid), polystyrene, nylon, polycarbonate and polysulfone.

28. The method of claim 26, wherein the arcuoid substrate is a fibre.

29. The method of claim 28, wherein the diameter of the fibre is between about 1 μm to about 1 mm.

30. The method of claim 28, wherein the diameter of the fibre is between about 50 μm to about 250 μm.

31. The method of claim 26, wherein the polymeric film is linked on the surface of the arcuoid substrate by deposition.

32. The method of claim 31, wherein the polymeric film is deposited with radiofrequency glow discharge.

33. The method of claim 26, wherein the polymeric film is a cross-linked polymeric film.

34. The method of claim 26, wherein the polymeric film is cross-linked to the surface of the arcuoid substrate.

35. The method of claim 33, wherein the polymeric film comprises at least one of a n-heptylamine monomer and an acetyldehyde monomer.

36. The method of claim 26, wherein the polymeric film comprises a reactive group.

37. The method of claim 26, wherein the thickness of the polymeric film is between about 2 nm to about 50 nm.

38. The method of claim 26, further comprising linking a biologically active entity to the polymeric film.

39. The method of claim 38, wherein the biologically active entity is linked to the polymeric film by an amide bond.

40. The method of claim 38, wherein the biologically active entity comprises a peptidic sequence.

41. The method of claim 40, wherein the biologically active sequence is at least one of a matrix protein, a portion of a matrix protein, a variant of a matrix protein, a denatured matrix protein, an enzyme and a growth factor.

42. The method of claim 38, wherein the biologically active entity is at least one of a RGD peptide and gelatin.

43. The method of claim 38, further comprising linking a spacer between the polymeric film and the biologically active entity, the spacer being linked to the polymeric film and to the biologically active entity.

44. The method of claim 43, wherein the spacer is linked to the polymeric film and to the biologically active entity by an amide bond.

45. The method of claim 43, wherein the spacer comprises at least one of a carboxymethyl dextran, a poly(ethylene oxide), a partially amino-functionalized dextran and heparin.

46. The method of claim 43, wherein the spacer comprises a carboxymethyl dextran.

47. The method of claim 46, wherein the molecular weight of the carboxymethyl dextran is between about 50 kDa to about 250 kDa.

48. The method of claim 46, wherein the molecular weight of the carboxymethyl dextran is about 70 kDa.

49. The method of claim 46, wherein the ratio of carboxylation of the carboxymethyl dextran is about 1:2.

50. A method for inducing angiogenesis, said method comprising the step of culturing endothelial cells on the bioactive composition of claim 1 for a time sufficient for spouting to occur and capillaries to be formed.
